(19) United States
(12) Patent Application Publication
(10) Pub. No.: US 2021/0008174 A1
(43) Pub. Date: Jan. 14, 2021

(54) Veterinary Composition Comprising Superoxide Dismutase and at Least One Hydrolysate of Proteins Rich in Bioassimilable Peptides

(71) Applicant: Matieres Premieres Internationales, Grasse (FR)

(72) Inventors: Cécile Destaing, Valbonne (FR); Pascal Renaglia, Biot (FR)

(21) Appl. No.: 17/040,209

(22) PCT Filed: Mar. 21, 2019
(86) PCT No.: PCT/FR2019/050650

§ 371 Date: Sep. 22, 2020

(30) Foreign Application Priority Data
Mar. 23, 2018 (FR) 1852515

(51) Int. Cl. A61K 38/44; A61K 9/00; A61K 38/01; A61K 9/48; A61P 25/20; A23K 20/189; A23K 20/147; A23K 50/40; A23K 10/22

(52) U.S. Cl. CPC A61K 38/446; A61K 9/0053; C12Y 115/01001; A61K 38/012; A61K 9/4866; A61K 9/4858; A23K 10/22; A61K 9/4825; A61P 25/20; A23K 20/189; A23K 20/147; A23K 50/40; A61K 9/485

(57) ABSTRACT

A veterinary composition comprising, in effective amounts, superoxide dismutase and at least one protein hydrolysate rich in bioassimilable peptides.

### TECHNICAL FIELD

The invention relates to the field of animal nutrition and/or veterinary medicine.

In particular, the present invention relates to a veterinary composition comprising, in effective amounts, superoxide dismutase (SOD) and at least one hydrolysate of proteins rich in bioassimilable peptides.

### STATE OF THE ART

Fear is considered to be a state of alert and agitation caused by a present or threatening danger (Sherman and Mills, 2008). Anxiety, for its part, is a response to a possible or imagined danger or to uncertainty (Sherman and Mills, 2008).

Just as in humans, fear and anxiety are considered in animals (in particular in domestic animals and especially in companion animals—such as dogs (Canis lupus familiaris) or cats (Felis silvestris catus) and new companion animals (NCA)—to be major emotional disorders.

Indeed, dogs experiencing these conditions may present behaviour disorders (also called behavioural disorders), namely undesired behaviours which may affect human-animal relations and unfortunately may cause reactions ranging from abandonment to euthanasia (Bamberger and Houpt, 2006; Casey, 2002).

Clomipramine is the only psychotropic authorised up to now for the treatment of anxiety in dogs with a slow action time (Sherman and Mil, 2008). A new gel for oral transmucosal absorption with dexmedetomidine (alpha-2 adrenergic agonist receptors) is the first treatment for dogs suffering from an aversion to noise (Korpivaara et al., 2017). Nevertheless, short action times (half-lives ranging from 0.5 to 3 hours), precautions for use, warnings, contraindications and other undesirable effects, as well as the requirement for issuing by veterinary prescription, are all impediments to its use (cf. Sileo Summary of Product Characteristics 2015).

In view of these difficulties and other disadvantages, various products/supplements possessing potential anxiolytic effects have been tested such as alpha-casozepine (Beata et al., 2007; Palestrini et al., 2010), L-theanine (Araujo et al., 2010), dog calming pheromone (Denenberg and Landsberg, 2008), and a combination of tryptophan and alpha-casozepine (Kato et al,. 2012). A fish hydrolysate has also been assessed with the aid of two different doses and is presented as having a certain effectiveness regarding reduction in the hyperactive response/reaction of dogs in response to the sound of thunder and for reduction in the secretion of cortisol (Landsberg et al., 2015). However, Landsberg et al. are only able to identify a reduction in blood cortisol levels and a reduction in the hyperactive reaction only in certain dogs, in response to an acute and short stress factor (in this case the sound of thunder) but do not give any indication as to the anxiolytic potential (prevention, regulation and/or treatment of anxiety) of the fish hydrolysate tested. In addition, the dogs tested in this study are all dogs belonging to the same species, namely beagles from experimental kennels, a canine population which cannot be considered to be representative.

Moreover, an anti-stress and anti-fatigue effect on healthy human individuals is reported following a double-blind randomised controlled clinical trial, against placebo, of a food supplement which can be administered per os comprising a melon juice concentrate rich in superoxide dismutase (EXTRAMEL®) (Milesi et al., 2009). This melon juice concentrate rich in superoxide dismutase has been validated for its antioxidant and anti-inflammatory properties (Vouldoukis et al., 2004; Muth et al., 2004).

In the veterinary field, a pilot study carried out on 24 horses showed antioxidant effects of superoxide dismutase on the blood and on muscle function with no side effects (Notin, 2010). Furthermore, still in the veterinary field, Lanes et al. (2010) observe that a melon pulp concentrate rich in superoxide dismutase makes it possible to obtain a reduction in certain markers of oxidative stress along the gastrointestinal tract in weaned piglets. However, Lanes et al. do not suggest in any way any correlation whatsoever between the administration per os of a melon pulp concentrate rich in superoxide dismutase and the prevention, regulation and/or treatment, in piglets, of fear and/or anxiety or at least a behaviour disorder, in particular associated with fear and/or anxiety.

The inventors have discovered that a veterinary composition combining superoxide dismutase and at least one protein hydrolysate rich in bioassimilable peptides (namely biologically assimilable by an animal organism) made it possible, successfully:
i) to prevent, regulate and/or treat, in animals, in particular in domestic animals (preferably in companion animals such as dogs, cats and/or new companion animals, preferably in companion animals such as dogs and/or cats):
   fear and/or anxiety, preferably anxiety, in particular in response to chronic mild stress factors and/or
   at least one behaviour disorder, preferably associated with fear and/or with anxiety, advantageously associated with anxiety, and
ii) to improve learning processes in animals, in particular in domestic animals, preferably in companion animals such as dogs, cats and/or NCAs, advantageously in companion animals such as dogs and/or cats.

### STATEMENT OF THE INVENTION

Consequently, the subject of the invention is a veterinary composition, which can preferably be administered orally, comprising, in effective amounts (or essentially consisting of, in effective amounts):
superoxide dismutase or at least one source of superoxide dismutase, and
at least one protein hydrolysate or at least one preparation based on at least one protein hydrolysate, said at least one protein hydrolysate advantageously being devoid of proteins, said at least one protein hydrolysate comprising a peptide fraction in which less than 1% by weight of the peptides have a molecular weight greater than or equal to 10,000 Da, preferably in which 100% by weight of the peptides have a molecular weight less than 10,000 Da, preferably less than 3,000 Da, advantageously less than or equal to 1,800 Da, in a preferred manner less than 1,800 Da.

Preferably, said peptide fraction represents at least 50% by weight, preferably more than 50% by weight, preferably at least 55% by weight, advantageously at least 60% by weight, in a preferred manner more than 60% by weight, relative to the total weight of said at least one protein hydrolysate.

According to a preferred embodiment, the veterinary composition according to the invention comprises at least one source of superoxide dismutase and at least one preparation based on at least one protein hydrolysate in a weight ratio (source of superoxide dismutase/preparation based on at least one protein hydrolysate) between 0.01:100 and 100:1, preferably between 1:100 and 1:10, preferably between 1:100 and 5:100, advantageously between 1:50 and 2:50.

Preferably, said superoxide dismutase is of plant origin, preferably of fruit origin, advantageously derived from Olea europeae, from Vitis vinifera and/or from at least one Cucurbitaceae such as Cucumis melo; preferably said superoxide dismutase consisting of the superoxide dismutase of Cucumis melo.

Preferably, said at least one protein hydrolysate is an animal protein hydrolysate, preferably a protein hydrolysate of at least one marine animal, preferably a fish protein hydrolysate, advantageously a protein hydrolysate of fish belonging to the Gadidae family.

The invention also relates to a nutritional composition, a compound feed for animals (such as a complete feed for animals or a supplementary feed for animals), a feed for animals which is aimed at specific nutritional objectives, comprising the veterinary composition according to the invention.

The subject of the invention is also a veterinary medicine comprising the veterinary composition according to the invention.

The subject of the present invention is also:
the veterinary composition according to the invention,
the nutritional composition, the compound feed for animals, or the feed for animals which is aimed at specific nutritional objectives, as defined previously, and/or
the veterinary medicine as defined previously,
   for its/their use(s) as a veterinary medicine, in particular in domestic animals, preferably in companion animals such as dogs, cats and/or NCAs, advantageously in companion animals such as dogs and/or cats.

Another subject of the invention relates to:
the composition according to the invention,
the nutritional composition, the compound feed for animals, or the feed for animals which is aimed at specific nutritional objectives, as defined previously, and/or
the veterinary medicine as defined previously,
   to prevent, regulate and/or treat, in animals, in particular in domestic animals, preferably in companion animals such as dogs, cats and/or NCAs, advantageously in companion animals such as dogs and/or cats:
fear and/or anxiety, preferably anxiety, in particular in response to chronic mild stress factors and/or
at least one behaviour disorder, preferably associated with fear and/or with anxiety, advantageously associated with anxiety.

Preferably, said at least one behaviour disorder is selected from: the act of attacking, aggressiveness, destroying, inappropriate elimination, the act of repeatedly licking a part of its body, the act of scratching itself, astasia, the act of trembling, the act of marking its territory, aberrant motor behaviour, abnormal feeding behaviour such as polyphagia or polydipsia, dysbasia, an abnormal sensory profile, an abnormal posture, an abnormal vocalisation, sleep disorders, loss of expression, loss of sociability and abnormal judgement of situations.

The subject of the invention is also:
the veterinary composition according to the invention,
the nutritional composition, the compound feed for animals, or the feed for animals which is aimed at specific nutritional objectives, as defined previously, and/or
the veterinary medicine as defined previously,
   for its/their use in the improvement of learning processes in animals, in particular in domestic animals, preferably in companion animals such as dogs, cats and/or NCAs, advantageously in companion animals such as dogs and/or cats.

According to a preferred embodiment, the veterinary composition according to the invention comprises (or essentially consists of):
a) from 0.5 to 100 IU, preferably from 20 to 80 IU, preferably from 40 to 65 IU, advantageously from 50 to 60 IU (for example 55 IU), of superoxide dismutase, and/or
b) from 10 to 1,000 mg, preferably from 200 to 800 mg, preferably from 400 to 600 mg, advantageously between 450 and 550 mg (for example 500 mg), of said at least one preparation based on said at least one protein hydrolysate, preferably said composition comprising a) and b) (or essentially consisting of a) and b)).

According to a preferred embodiment, said composition comprises (or essentially consists of):
a) from 0.1 to 20 mg, preferably from 5 to 15 mg, preferably from 9 to 13 mg, advantageously from 10 to 12 mg (for example 11 mg), of a source of superoxide dismutase, and/or
b) from 10 to 1,000 mg, preferably from 200 to 800 mg, preferably from 400 to 600 mg, advantageously between 450 and 550 mg (for example 500 mg), of said at least one preparation based on said at least one protein hydrolysate,
   preferably said composition comprising a) and b) (or essentially consisting of a) and b)).

The subject of the invention is also the veterinary composition according to the invention, said composition being administered to the animal in the form of at least one dose (preferably encapsulated within a capsule), preferably in the form of a plurality of doses, said at least one dose comprising (or essentially consisting of):
a) from 0.5 to 100 IU, preferably from 20 to 80 IU, preferably from 40 to 65 IU, advantageously from 50 to 60 IU (for example 55 IU), of superoxide dismutase per kilogram of body weight of said animal, and/or
b) from 10 to 1,000 mg, preferably from 200 to 800 mg, preferably from 400 to 600 mg, advantageously between 450 and 550 mg (for example 500 mg), of said at least one preparation based on said at least one protein hydrolysate per kilogram of body weight of said animal,
   preferably said dose comprising a) and b) (or essentially consisting of a) and b)).

According to an embodiment, said dose comprises (or essentially consists of):
a) from 0.1 to 20 mg, preferably from 5 to 15 mg, preferably from 9 to 13 mg, advantageously from 10 to 12 mg (for example 11 mg), of a source of superoxide dismutase per kilogram of body weight of said animal, and/or
b) from 10 to 1,000 mg, preferably from 200 to 800 mg, preferably from 400 to 600 mg, advantageously between 450 and 550 mg (for example 500 mg), of said at least one preparation based on said at least one protein hydrolysate per kilogram of body weight of said animal,
   preferably said dose comprising a) and b) (or essentially consisting of a) and b)).

Another subject of the invention relates to a superoxide dismutase, preferably of plant origin, preferably of fruit origin, advantageously derived from Olea europeae, from Vitis vinifera and/or from at least one Cucurbitaceae such as Cucumis melo; in a preferred manner consisting of the superoxide dismutase of Cucumis melo, for:
i) its use for preventing, regulating and/or treating, in animals, in particular in domestic animals, preferably in companion animals such as dogs, cats and/or NCAs, advantageously in companion animals such as dogs and/or cats:
   fear and/or anxiety, preferably anxiety, and/or
   at least one behaviour disorder, preferably associated with fear and/or with anxiety, advantageously associated with anxiety, and/or
ii) for its use in the improvement of learning processes in animals, in particular in domestic animals, preferably in companion animals such as dogs, cats and/or NCAs, advantageously in companion animals such as dogs and/or cats.

The composition according to the invention not only has properties which are advantageous in terms of effectiveness and rapidity of action, but can, moreover, be administered to an animal without contraindications and without familiarisation, for example as a supplement to conventional veterinary medicine(s). Moreover, this composition also has the advantage of being “multi-species”.

According to an embodiment, the composition according to the invention—and the nutritional compositions, compound feeds for animals, supplementary feeds for animals, feeds for animals aimed at specific nutritional objectives and veterinary medicines containing them—comprises at least one excipient and/or additive which is acceptable from the feeding and/or pharmaceutical point of view and which is added with a view to obtaining the desired dosage form, which can preferably be administered orally. Said at least one excipient and/or additive is/are, for example, selected from binders, lubricants, sweeteners, diluents, coating agents, and flavours (natural or artificial).

A subject of the invention is also solid dosage forms (such as capsules, tablets, chewable tablets, pastilles, mixtures of solid and divided particles (for example a non-effervescent powder etc.)) and liquid dosage forms (solutions, drinkable suspensions, gels, syrups, liquid compositions which can be encapsulated in soft or hard capsules etc.) comprising, essentially consisting of, or consisting of the composition according to the invention, said liquid dosage forms advantageously being packaged in sachets (preferably airtight) or in sticks (preferably airtight), in particular for reasons of convenience of administering and to promote compliance with the treatment/cure.

It should be noted that liquid dosage forms (more simply called “liquid forms”) are understood, broadly, to be any composition or formulation that can take the form of the receptacle which contains it (for example the form of the sachet or the stick mentioned above) but whose volume is fixed. Thus, and as indicated above, these liquid forms include/encompass especially solutions, drinkable suspensions, gels and syrups.

Preferably, the composition according to the invention can be administered orally. Advantageously, the composition according to the invention is in solid form. According to a preferred embodiment, the composition according to the invention is presented in the form of a chewable tablet or a powder (pulverulent form) encapsulated in a capsule, advantageously in the form of a powder encapsulated in a capsule.

According to a preferred embodiment, and in particular when the composition according to the invention is presented in the form of a powder encapsulated in a capsule, said composition comprises at least one excipient selected from the following: microcrystalline cellulose, magnesium stearate, silicon dioxide, beer yeast, colloidal silica. Advantageously, the composition according to the invention comprises, as excipient, microcrystalline cellulose (microcellulose).

### DEFINITIONS

Superoxide dismutase (SOD). SOD is a metalloprotein that possesses an enzymatic activity which catalyses the dismutation of superoxide anions into dioxygen and hydrogen peroxide as follows:

$$\begin{matrix}{O}_{2}^{\text{?}} + {M}^{2 +} \to {O}_{2} + {M}^{1 +} \\ {O}_{2}^{\text{?}} + {M}^{1 +} + 2 {H}^{+} \to {H}_{2} {O}_{2} + {M}^{2 +}\end{matrix} \} 2 {O}_{2}^{\text{?}} + 2 {H}^{+} \to {H}_{2} {O}_{2} + {O}_{2}$$

$$\text{?} \text{indicates text missing or illegible when filed}$$

Studies have shown that SOD is a ubiquitous enzyme (namely present in almost all aerobic organisms), different isoforms of this metalloprotein having been identified later, each characterized by

a metal ion located at its active site (metal cofactors Cu, Zn, Fe or Mn).

As indicated above, these metalloproteins are present in almost all organisms which consume oxygen, in aerotolerant anaerobes and some strict anaerobes. Only the iron form (Fe SOD) is present only in prokaryotic cells and some species of plants.

The different isoforms of SOD are determined by their sensitivity:
Cu/Zn SOD is inhibited by cyanide and by H2O2,
Fe SODs are inhibited by H2O2 and weak concentrations of SDS.

The O2⁻ radical can traverse plasma membranes with difficulty, and consequently it must be detoxified in the same compartment where it was formed. This phenomenon explains the existence of different cytosolic, mitochondrial and extracellular isoforms of superoxide dismutase.

Cytosolic CuZn SOD is a soluble, generally very stable enzyme, mainly present in the cytosol of eukaryotic cells (exists in some bacteria), but also in the chloroplasts of plant cells. It is a homodimeric intracellular protein, and its two subunits each contain a Cu²⁺ ion and a Zn²⁺ ion and their molecular weight varies from 16 to 19 kDa. The spatial organisation of the electrostatic field at the surface of the Cu/Zn superoxide dismutase reveals a positively charged region, thus creating a gulf which leads the superoxide radicals towards the copper ion in order to cause an effective collision.

The extracellular form is a tetrameric glycosylated Cu/Zn superoxide dismutase. It is present in mammals, some plants, and also some prokaryotes. Within the bacteria, this enzyme is periplasmic, and is released into the extracellular medium only following an osmotic shock. Its presence is necessary to protect the cell against the numerous extracellular sources of O2⁻.

Mn SOD is a homodimeric mitochondrial enzyme of 40 to 46 kDa in eukaryotes, in the tetrameric form of 110 to 140 kDa in prokaryotes.

Fe SOD, non-existent within animal tissues, as indicated above, has significant homologies, of amino acid sequences and of structure, with Mn SOD. Being homodimeric, each of its subunits contains an iron atom and has a molecular weight of 23 kDa, and it can also be encountered in tetrameric form in mammals.

In physiological conditions, the organism permanently produces oxidising molecules, called free radicals (FR), such as reactive oxygen species (ROS). As is known to the person skilled in the art, the oxidising power of free radicals (FR) is strictly controlled by antioxidants forming a fragile equilibrium. In certain conditions (stress, pollution, etc.), this equilibrium can easily be disrupted in favour of a significant production of free radicals which are linked or not linked to a lowering of antioxidant activity, which leads to oxidative stress (also called “oxidant stress” in the literature).

As is known in the state of the art, the increase in the level of free radicals brings about much irreversible damage to the cells of the organism, such as peroxidation of the membrane lipids, mitochondrial degradation, protein oxidation and alterations to the DNA. Indeed, the free radicals may induce mutagenic effects or the stopping of DNA replications (alteration of bases, DNA-protein bridging, strand breakages, cross-linking). OH radicals are the main elements responsible for wastage. They are derived from the Fenton reaction, in the presence of ferrous iron chelated to certain amino acids or to the phosphate groups of DNA. Being very reactive, they react immediately on their sites of formation, that is to say the nucleotide part with which they are directly in contact. These active forms of oxygen are also responsible for enzymatic inactivations, a fragmentation of the macromolecules, formation of dimers or protein aggregates in the cytoplasmic membranes.

Due to the creation of abnormal biological molecules and the overexpression of certain genes, oxidative stress is the basic initial cause of numerous pathologies such as cancers, pulmonary oedema, acute pulmonary distress syndrome, amyotrophic lateral sclerosis, pro-inflammatory and cardiovascular diseases, neurological disorders, fibroses, diabetes, cellular aging, Alzheimer's disease, rheumatisms and also accelerated aging.

Oxidative stress is also one of the factors which potentiate the genesis of plurifactorial diseases such as diabetes, Alzheimer's disease, rheumatisms and cardiovascular diseases.

Similarly in numerous infection situations, the reduction in antioxidant capacities - thus facilitating oxidative stress—reduces immune defences.

Thus, the enzymatic activity of SOD makes it possible to reduce the amount of superoxide anions present in the organism and, as a consequence, to prevent and/or inhibit oxidative damage which can be caused by oxidising molecules derived from the superoxide anion, such as the hydroxyl or peroxynitrite radical.

Preferably, the veterinary composition according to the invention comprises superoxide dismutase of plant origin, preferably superoxide dismutase derived from Olea europeae (as mentioned in patent application FR-A-3003165, the content of which is incorporated by reference), from Vitis vinifera (as mentioned in patent application FR-A-3003164, the content of which is incorporated by reference) and/or from Cucurbitaceae such as melon (Cucumis melo); in a preferred manner, said superoxide dismutase is derived from Cucumis melo.

Preferably, the veterinary composition according to the invention comprises at least one source of superoxide dismutase, preferably of plant origin (advantageously of fruit origin and in a preferred manner derived from at least one Cucurbitaceae such as Cucumis melo). According to a preferred embodiment, said at least one source of superoxide dismutase consists of at least one preparation (for example at least one extract) comprising superoxide dismutase, preferably of plant origin (advantageously of fruit origin and in a preferred manner derived from at least one Cucurbitaceae such as Cucumis melo). Preferably, said at least one preparation is a preparation based on Olea europeae, Vitis vinifera and/or at least one Cucurbitaceae such as Cucumis melo (advantageously based on Cucumis melo); said preparation advantageously containing superoxide dismutase in an amount of at least 100 IU/g of dry matter of said preparation (for example of the extract), preferably of at least 1000 IU/g of dry matter of said preparation, preferably at least 3000 IU/g of dry matter of said preparation and, preferably, at least 5000 IU/g of dry matter of said preparation.

In biochemistry, the enzymatic unit (symbol U or IU) is a unit of enzyme activity representing the amount of enzymes necessary to treat one micromole of substrate in one minute under operating conditions (pH, temperature, solution parameters). The value generally corresponds to conditions which are optimal for enzymatic activity, but the values are sometimes normalised at 30° C. in order to allow comparisons between enzymes.

The enzymatic unit is associated with the katal, the unit of the International system of enzyme activity, by the relationship: 1 kat=6×10⁷ U.

According to one embodiment, use is made, for the purposes of the present invention, of an extract (preferably a protein extract, advantageously hydrosoluble), such that it can be obtained from Cucumis melo presenting an ethylene production plateau after the ethylene crisis for at least five days, preferably for at least seven days (that is to say that ethylene production presents a plateau that can be at least five days and preferably at least seven days). As is known in the prior art, the ethylene crisis in Cucumis melo is highly significant and a short time afterwards, the fruit starts to become disorganised and its market value decreases. In contrast, in the case of Cucumis melo which are capable of providing the abovementioned protein extract, the emission of ethylene presents a stable plateau preferably for at least five days after the ethylene crisis and even more advantageously for at least seven days. The abovementioned protein extract can be obtained in particular from the 95LS444 cell line or from one of the hybrid lines derived from 95LS444 and more particularly from the Vauclusien, Clipper and Supporter type commercial varieties. Indeed, starting from the 95LS444 line of Cucumis melo whose seeds were deposited, in accordance with the Budapest Treaty, in the NCIMB (National Collection of Industrial and Marine Bacteria-ABERDEEN AB2 1RY (Scotland—GB) 23 St. Machar Drive) collection on 19 Jul. 1990, under number 40310, it is possible to obtain, by hybridisation, other varieties of Cucumis melo for example varieties of the Vauclusien, Clipper and Supporter type presenting the same characteristics making it possible to lead to the abovementioned protein extracts.

According to an embodiment, the composition according to the invention comprises, as source of SOD (for example as the sole source of SOD), a melon juice concentrate which is freeze-dried and coated with at least one oil, for example coated with palm oil.

According to an embodiment, the composition according to the invention comprises, as source of SOD (for example as the sole source of SOD), the product EXTRAMEL® microgranules (M and/or S form(s)). This product is a freeze-dried extract of melon juice obtained by physical treatment (milling of the melon, recovery of the pulp, centrifugation, filtering, freeze-drying) of a specific variety of melon (non-GMO, Clipper variety, originating from one of the hybrid lines derived from the abovementioned 95LS444 cell line) (cf. FR-B-2716884; the content of which is incorporated by reference), which contains enzymatic antioxidants, mainly SOD (90 IU/mg), measured according to the Oberley and Spitz method, and, to a lesser extent, catalase (10 IU/mg), which was determined according to the Clairbone method. Powdered melon juice, coated with palm oil, which contains 14 IU of SOD/mg of powder, is called EXTRAMEL® microgranules and is manufactured by Bionov Co. (France).

More precisely, the product EXTRAMEL® microgranules is obtained by coating the melon pulp concentrate (Cucumis melo) (20%) with palm oil, a vegetable fat (80%). The product is a natural and effective source of natural antioxidants and in particular of antioxidant enzymes such as superoxide dismutase (SOD) and catalase.

The SOD content in the product EXTRAMEL® microgranules is 14,000 IU/g.

According to a preferred embodiment, the composition according to the invention comprises, as a source of SOD (and advantageously as the sole source of SOD), the product SOD B Primo-antioxidant® (5 IU/mg), in its/their M and/or S form(s), advantageously in its M form. This product, manufactured by Bionov Co. (France), is a concentrate of Cucumis melo L juice. (non-GMO proprietary variety of Cantaloup melon 5 to 10 times more concentrated in SOD than a classic variety) which is freeze-dried and coated with palm oil and contains 5 IU of SOD/mg powder.

According to a particular embodiment, the veterinary composition according to the invention comprises a mixture of superoxide dismutases of plant origin, primarily consisting of three superoxide dismutases: one manganese superoxide dismutase, one copper and zinc superoxide dismutase and one iron superoxide dismutase present in at least two isoforms, the first iron superoxide dismutase isoform with a molecular weight between 28,000 and 36,000 Da, the second iron superoxide dismutase isoform with a molecular weight between 75,000 and 85,000 Da, it being possible to obtain said mixture from an extract of the Fl hybrid variety of Cucumis Melo MA 7950 or one of its cells grown in vitro or by transfer and expression of the genes of these SODs in prokaryotic or eukaryotic cells.

In this particular embodiment, said mixture has a total SOD activity greater than or equal to 130 U/mg of said mixture.

Still in this particular embodiment, the first iron superoxide dismutase isoform has a molecular weight of approximately 32,200 Da.

Still in this particular embodiment, the second iron superoxide dismutase isoform has a molecular weight of approximately 79,800 Da.

Still in said particular embodiment, the cumulative SOD activity of the two iron superoxide dismutase isoforms is between 20% and 26%, advantageously between 22% and 26% of total SOD activity of the mixture.

Still in said particular embodiment, the cumulative SOD activity of the two iron superoxide dismutase isoforms is between 20% and 26% of the total SOD activity of the mixture, the activity of the copper and zinc superoxide dismutase is between 60% and 70% of the total SOD activity of the mixture, and the activity of the manganese superoxide dismutase is between 7 and 12% of the total SOD activity of the mixture.

Still in said particular embodiment, the manganese superoxide dismutase has a molecular weight between 70,000 and 90,000 Da and the copper and zinc superoxide dismutase has a molecular weight of between 27,000 and 33,000 Da.

According to a preferred aspect of this particular embodiment, it is possible to obtain the mixture of the three superoxide dismutases by milling or pressing in an aqueous medium, preferably at a pH of 5 to 9, of the Fl hybrid variety of Cucumis Me/o MA 7950 or of its cells cultivated in vitro or by transfer and expression of the genes of these SODs in prokaryotic or eukaryotic cells then recovery of the supernatant and purification by chromatography, in particular by IMAC chromatography.

As indicated in patent application WO 2016/128531 (the content of which is also incorporated herein by reference), the Fl hybrid variety of Cucumis melo MA 7950, whose seeds were deposited, in accordance with the Budapest Treaty, in the NCIMB (National Collection of Industrial and Marine Bacteria-ABERDEEN AB21 9YA (Scotland—GB) Ferguson Building Craibstone Estate Bucksburn) on 8 Jul. 2013, under number NCIMB 42154, possesses characteristics which are unique in terms of its appearance, its resistance to stress and its SOD composition. This SOD mixture possesses antioxidant properties which are superior to other SOD mixtures derived from other plant sources, and especially derived from other varieties of melon.

Protein hydrolysate rich in bioassimilable peptides (namely biologically assimilable by the animal organism). The veterinary composition according to the invention comprises at least one protein hydrolysate rich in bioassimilable peptides, namely preferably devoid of proteins and whose peptide fraction has the following molecular profile:
less than 1% by weight of the peptides have a molecular weight greater than or equal to 10,000 Da, and, preferably
100% by weight of the peptides have a molecular weight less than 10,000 Da, preferably less than 3,000 Da, advantageously less than or equal to 1,800 Da, in a preferred manner less than 1,800 Da.

In other words, the protein hydrolysate which can be used in the veterinary composition according to the invention can be referred to as a protein hydrolysate rich in bioassimilable peptides (namely biologically assimilable by an animal organism), owing to the relatively low molecular weight of the peptides constituting the peptide fraction of said hydrolysate.

According to a preferred embodiment, said peptide fraction represents at least 50% by weight, preferably more than 50% by weight, preferably at least 55% by weight, advantageously at least 60% by weight, in a preferred manner more than 60% by weight, relative to the total weight of said at least one protein hydrolysate.

According to an embodiment of the invention, the molecular weight distribution (expressed as a weight percentage relative to the total weight of the peptide fraction of the hydrolysate) of the protein hydrolysate rich in bioassimilable peptides which can be used in the veterinary composition according to the present invention has the profile presented in table 1 below:

TABLE 1

| | |
|---|---|
| MW(*) ≥ 1800 daltons | Absence |
| 1800 ≤ MW ≥ 600 daltons | Approximately 35% (for example 35%) |
| 600 ≤ MW ≥ 300 daltons | Approximately 30% (for example 30%) |
| ≤300 daltons | Approximately 35% (for example 35%) |
| Residual proteins | Absence |

(*)MW: molecular weight

Preferably, the molecular weight distribution (expressed as a weight percentage relative to the total weight of the peptide fraction of the hydrolysate) of the abovementioned protein hydrolysate rich in bioassimilable peptides has the profile defined in the following table 2:

TABLE 2

| | |
|---|---|
| MW(*) > 1800 daltons | Absence |
| 1800 < MW > 600 daltons | Approximately 35% (for example 35%) |
| 600 < MW > 300 daltons | Approximately 30% (for example 30%) |
| <300 daltons | Approximately 35% (for example 35%) |
| Residual proteins | Absence |

(*)MW: molecular weight

Preferably, the protein hydrolysate which can be used in the veterinary composition according to the invention comprises the following amino acids: aspartic acid, threonine, serine, glutamic acid, proline, glycine, alanine, cystine, methionine, isoleucine, leucine, tyrosine, phenylalanine, lysine, histidine, arginine and tryptophan.

The protein hydrolysate which can be used in the composition according to the invention can be a plant protein hydrolysate or an animal protein hydrolysate. Advantageously, it is an animal protein hydrolysate. Preferably, said protein hydrolysate is an animal/marine animal protein hydrolysate.

According to a preferred embodiment, the abovementioned protein hydrolysate is different from a milk protein hydrolysate (for example a lactoserum hydrolysate such as a bovine lactoserum hydrolysate).

The protein hydrolysate of interest is obtained by hydrolysis, advantageously by enzymatic hydrolysis said to be “controlled” or “managed”, of at least one source of proteins of at least one type. The hydrolysis is continued until the hydrolysate is obtained whose peptide fraction corresponds to the molecular profile as defined previously. Stoppage of the enzymatic hydrolysis can be obtained by inactivation of the proteases by raising the temperature of the reaction mixture, preferably to a temperature not exceeding 100° C., especially between 85 and 95° C., preferably around 90° C. This operation is generally conducted for a time ranging from 5 to 20 minutes.

According to a preferred embodiment, the composition according to the invention comprises a preparation based on at least one protein hydrolysate rich in bioassimilable peptides, and more particularly based on at least one fish hydrolysate, namely comprising, in addition to said protein hydrolysate rich in bioassimilable peptides, at least one suitable excipient (for example selected from maltodextrin and colloidal silica).

Animal/marine animal protein hydrolysate. Preferably, said animal/marine animal protein hydrolysate is a fish, mollusc and/or crustacean protein hydrolysate. According to an embodiment, this protein hydrolysate comes from tissues of marine animals (in particular fish, molluscs and/or crustaceans) which are rich in collagen.

According to a preferred embodiment, the veterinary composition according to the invention comprises a fish protein hydrolysate, advantageously a protein hydrolysate of fish belonging to the Gadidae family, cold-water fish. Advantageously, this fish protein hydrolysate is obtained from a selection of one or more (preferably several) eviscerated fish from the Gadidae family which are fished in the North Atlantic: Cusk, Cod, Coley, Pollack, Ling, Eling, Salt Cod, Hake, Young Hake, Whiting, Haddock, Grenadier, Pout, Loach, Conger Eel.

Preferably, the fish protein hydrolysate which can be used in the veterinary composition according to the invention comprises a peptide fraction comprising peptides and amino acids.

According to a preferred embodiment of the invention, proteins represent less than 1% by weight, preferably less than 0.5% by weight of the peptide fraction of the fish protein hydrolysate. Particularly preferably, the fish protein hydrolysate is devoid of proteins (residual proteins).

The peptide fraction of the fish protein hydrolysate used in the veterinary composition according to the invention is, for example, the fish protein hydrolysate which is the subject of French patent application FR-A-3036923, the content of which is incorporated herein by reference. Thus, as described in this patent application, the peptide fraction of the fish protein hydrolysate (expressed as a weight ratio relative to the total weight of the peptide fraction) has the following molecular profile:
less than 1% of the molecules have a molecular weight greater than or equal to 10,000 Da, and
60 to 99% of the molecules have a molecular weight less than 1,500 Da.

As indicated in FR-A-3036923, this molecular profile of the peptide fraction is typically determined by high performance liquid chromatography coupled with mass spectrometry, on a silica gel separation column of the Shodex KW-802.5 type calibrated beforehand on reference molecules.

According to a preferred embodiment of the invention, and still as described in French patent application FR-A-3036923, the molecular profile of the peptide fraction of the fish protein hydrolysate which can be used in the veterinary composition according to the invention has the following distribution (expressed as a weight ratio relative to the total weight of the peptide fraction of the hydrolysate):
10 to 20% of the molecules have a molecular weight between 1,500 and 5,000 Da
35 to 45% of the molecules have a molecular weight between 500 and 1,500 Da
15 to 25% of the molecules have a molecular weight between 300 and 500 Da, and
19 to 29% of the molecules have a molecular weight less than 300 Da.

According to an embodiment of the invention, the molecular weight distribution (expressed as a weight percentage relative to the total weight of the peptide fraction of the hydrolysate) of the fish protein hydrolysate which can be used in the veterinary composition according to the present invention has the profile presented in table 3 below:

TABLE 3

| | |
|---|---|
| MW(*) ≥ 1800 daltons | Absence |
| 1800 ≤ MW ≥ 600 daltons | Approximately 35% (for example 35%) |
| 600 ≤ MW ≥ 300 daltons | Approximately 30% (for example 30%) |
| ≤300 daltons | Approximately 35% (for example 35%) |
| Residual proteins | Absence |

(*)MW: molecular weight

Preferably, the molecular weight distribution (expressed as a weight percentage relative to the total weight of the peptide fraction of the hydrolysate) of the abovementioned fish protein hydrolysate has the profile defined in the following table 4:

TABLE 4

| | |
|---|---|
| MW(*) > 1800 daltons | Absence |
| 1800 < MW > 600 daltons | Approximately 35% (for example 35%) |
| 600 < MW > 300 daltons | Approximately 30% (for example 30%) |
| <300 daltons | Approximately 35% (for example 35%) |
| Residual proteins | Absence |

(*)MW: molecular weight

According to an embodiment, the fish protein hydrolysate comprises at least one peptide chosen from IGP (isoleucine-glycine-proline), LGP (leucine-glycine-proline), VY (valine-tyrosine) and RP (arginine-proline).

According to an embodiment, the fish protein hydrolysate which can be used in the veterinary composition according to the invention comprises up to 25% and, preferably, up to 20% (for example less than 20%) of free amino acids, relative to the number of total amino acids.

Preferably, the fish proteins (namely the raw material), are derived from tissues of at least one type of fish of the Gadidae family (preferably of at least two types of fish of the Gadidae family).

According to an embodiment, the fish protein hydrolysate which can be used in the veterinary composition according to the invention comprises:
(i) the peptide fraction, which represents from 60 to 80% by weight of the total weight of dry extract of said hydrolysate,
(ii) a lipid fraction, which represents from 5 to 17% by weight of the total weight of dry extract of said hydrolysate, and
(iii) a mineral fraction, which represents from 5 to 23% by weight of the total weight of dry extract of said hydrolysate.

The fish protein hydrolysate which can be used in the veterinary composition according to the invention can have at least one of the following characteristics:
a total nitrogenous material content (estimated according to the formula N×6.25) between 41 and 91% by weight relative to the total weight of the dry extract;
an amino nitrogen/total nitrogen ratio between 25 and 40%, especially between 30 and 34%;
a free amino acids content between 15 and 35% of the total nitrogenous material.

According to one embodiment, said hydrolysate comprises a carbohydrate content less than 0.2% and especially less than 0.1%, especially less than 0.05% and particularly preferably less than 0.002% by weight relative to the total weight of dry extract of the hydrolysate. In some embodiments, the hydrolysate of the invention is devoid of carbohydrates.

The fish protein hydrolysate which can be used within the veterinary composition according to the invention can be associated with one or more supplementary, edible compounds chosen for example from an oil (plant and/or fish), a vitamin (like vitamin B1), or a mineral element. It is thus possible to use a purified fish flesh oil. Preferably an oil rich in omega-3 preferably containing at least 25% omega-3 will be chosen.

Preferably, the mineral element is at least one oligoelement like copper, for example. The supplementary compounds optionally added to the hydrolysate of the invention are advantageously chosen from the compounds which can be consumed by animals, especially by companion animals or farm animals, and more preferably by dogs (typically Canis lupus familiaris) or cats (Fells silvestris catus). The nutritional composition moreover can include ingredients which are suitable for use as a nutritional supplement such as binders, pulverulent carriers (like maltodextrin), flavouring agents, preservatives or colorants.

As indicated previously, according to a preferred embodiment, the composition according to the invention comprises a preparation based on at least one protein hydrolysate rich in bioassimilable peptides, and more particularly based on at least one fish hydrolysate, namely comprising, in addition to said protein hydrolysate rich in bioassimilable peptides, at least one excipient (for example selected from: maltodextrin and colloidal silica). According to a particularly preferred embodiment, this preparation based on at least one protein hydrolysate rich in bioassimilable peptides (and more particularly based on fish protein hydrolysate) is GABOLYSAT® (and more particularly GABOLYSAT® PTP), the description of which is presented hereinafter (table 5):

TABLE 5

| | |
|---|---|
| Common name | protein hydrolysate of fish of |
| | the Gadidae family |
| Form and colour | powder, pale grey colour with |
| | fish odour |
| Excipients | maltodextrin, colloidal silica |
| Route of administration | oral |

### MANUFACTURING METHOD

### Method of Managed Enzymatic Hydrolysis

Production of small peptides with molecular weights less than 1,800 daltons.

RAW MATERIAL ORIGINS

Selection of eviscerated fish from the Gadidae family which are fished in the North Atlantic: Cusk, Cod, Coley, Pollack, Ling, Eling, Salt Cod, Hake, Young Hake, Whiting, Haddock, Grenadier, Pout, Loach, Conger Eel.

### COMPOSITION OF PEPTIDES BY MOLECULAR WEIGHTS (MW) (±5)

TABLE 6

| | |
|---|---|
| MW > 1800 daltons | Absence |
| 1800 < MW > 600 daltons | 35% |
| 600 < MW > 300 daltons | 30% |
| <300 daltons | 35% |
| Residual proteins | Absence |

Method of obtaining fish protein hydrolysate. When a fish protein hydrolysate is used in the veterinary composition according to the invention, the obtaining method described on page 8, line 24 to page 11, line 14 of French patent application FR-A-3036923 can be envisaged. This obtaining method—described on page 8, line 24 to page 11, line 14 of French patent application FR-A-3036923—is incorporated into the present patent application by reference. However, for the sake of completeness, this is reproduced hereinafter.

The fish protein hydrolysate of interest is obtained by hydrolysis, preferably by enzymatic hydrolysis, advantageously by enzymatic hydrolysis said to be “controlled” or “managed”, of at least one source of proteins of at least one type (or of at least one species) of fish.

Preferably, tissues of at least one type of fish of the Gadidae family (preferably at least two types of fish of the Gadidae family) are used as a source of fish proteins (corresponding to the raw material). Whole fish or certain tissues of fish are used. It is also possible to use coproducts derived from the fish industry as raw material.

Preferably, the fish are eviscerated (or gutted), that is to say that their abdominal wall has been opened longitudinally, the viscera removed and the abdominal cavity cleaned. The fish can be decapitated. Water in the amount of 20 to 25% approximately by weight, relative to the total weight of the raw material, is advantageously added to this raw material. Acidified water, having a pH between 4.5 and 6, can be used.

The hydrolysate according to the invention is thus the result of treatment during the course of which some peptide bonds of the proteins are broken. The method according to the invention is characterised in that it comprises:
1) Optionally a step for milling said at least one source of fish proteins.
2) A step for enzymatically hydrolysing the source of fish proteins, preferably at a temperature ranging from 45° to 65° C. This step is typically performed with stirring. Advantageously the hydrolysis is performed at a constant pH ranging from 4.5 to 6, generally for a duration of 2 to 6 hours.

The hydrolysis is conducted by controlled (or managed) enzymatic digestion under the action of endogenous and exogenous protease(s). “Endogenous protease” is understood to mean any protease existing naturally in the source of fish proteins which is used (typically any protease which is naturally contained in the fish flesh used).

“Exogenous protease” is understood acording to the invention to mean any exogenous enzyme, that is to say added to the souce of fish proteins, and capable of hydrolysing the proteins of the raw materials selected which are subjected to the hydrolysis treatment. The proteases used must be compatible with feeding use of the hydrolysate in animals and especially in dogs or cats. It is especially possible to use one or more proteases of marine or bacterial origin. Preferably, at least one natural fish enzyme, or a mixture of natural fish enzymes (that is to say existing naturally in fish) is used as an exogenous protease. The following enzymes or mixtures of enzymes can be used: extract of fish intestine mucosa, pancreatic extract of fish, chymosin, trypsin, chymotrypsin, papain, alone or as mixture(s).

Typically, the enzymes (proteases) or the mixture of exogenous enzymes are added after the heating step. Hydrolysis is continued until the hydrolysate corresponding to the molecular profile as defined previously is obtained. Stoppage of the enzymatic hydrolysis can be obtained by inactivation of the proteases by raising the temperature of the reaction mixture, to a temperature not exceeding 100° C., especially between 85 and 95° C., preferably around 90° C. This operation is generally conducted for a time ranging from 5 to 20 minutes.
3) A step of separating the obtained protein hydrolysate from the rest of the reaction mixture derived from step 2).

This separation can be performed by filtration (for example on a filter of around 400 pm) and/or by centrifugation. The centrifugation can be performed at a speed of between 4,000 and 7,000 rotations per minute (revolution by minute or rpm). The pellet obtained is then eliminated.

Preferably, the separation of the protein hydrolysate is performed by filtering of the reaction mixture followed by centrifugation. The filtering of the reaction medium makes it possible to eliminate the solid matter.
4) Advantageously, a step of dehydrating the hydrolysate is carried out after step 3). This dehydration step is generally performed by concentration under vacuum. It typically makes it possible to obtain a paste, preferably containing at least 55% of dry extract.
5) Optionally a step of drying the hydrolysate at a low temperature, by freeze-drying or by spray-drying, can be carried out.

Recovery of the hydrolysate in powder form can involve operations known to the person skilled in the art such as: concentration under vacuum, drying at low temperature, milling and others.

The hydrolysate is thus recovered in the form of a powder which preferably contains 15% or less, especially 10% at least, for example between 5 and 10% and more preferably 5% or less by weight of moisture, relative to the total weight of the hydrolysate in powder form. Preferably, drying takes place by spray drying. The protein hydrolysate is then typically pulverised in a vessel in which the air has previously been heated in such a way that the water evaporates.

The powder obtained is separated from the water vapour and collected at the end of the drying step.
6) Optionally, a step of processing the protein hydrolysate, where appropriate in the form of a paste, or a powder, after carrying out step 4) and/or 5). The hydrolysate can be processed in the form of a paste concentrated to at least 55% of dry extract. It can also be processed in the form of a powder containing 15% or less, especially 10% or less, for example from 5 to 10% and preferably 5% or less by weight of residual moisture, in relation to the total weight of the hydrolysate in powder form. These hydrolysates can be incorporated in feeds (especially prior to extrusion) or in nutritional compositions (i.e.: nutritional supplements).

Peptide fraction. “Peptide fraction” is understood to mean the part of the protein hydrolysate comprising nitrogenous compounds consisting of amino acids (thus including peptides and free amino acids). These compounds are water-soluble molecules.

Peptide. “Peptide” is understood to mean a polymer comprising at least 2 amino acids bonded to each other by peptide bonds. Typically, a peptide comprises fewer than 100 amino acids and possesses a molecular weight generally less than 11,000 Da, preferably less than 10,000 Da.

Protein. The term “protein” refers to a polypeptide comprising one or more peptide chains and possessing a three-dimensional organisation in space. Typically, one protein comprises at least 120 amino acids and possesses a molecular greater than 15,000 Daltons (Da).

Protide. The term protide takes, for the purposes of the present application, its meaning accepted in biochemistry, namely accepted in biochemistry and in nutrition. It denotes amino acids and all their oligomers and polymers, namely: oligopeptide, dipeptide, tripeptide, tetrapeptide, pentapeptide, octapeptide, nonapeptide, decapeptide, polypeptides and proteins.

Domestic animal. A domestic animal is an animal living in people's homes or in their vicinity, which is raised and fed there, which breeds in captivity and which is altered compared to the wild form living in nature and which receives protection from humans in exchange for its produce (production animal or farm animal) or simply for its presence, for its beauty, or for pleasure (songbirds, carrier pigeons . . . ). By way of example, dogs (Canis lupus familiaris), cats (Fells silvestris catus) and horses (Equus ferus caballus or Equus caballus) are domestic animals.

It should be noted that the domestic animal is defined, in French law, as an animal belonging to “a species which has been the subject of continuous and constant pressure of selection (that is to say which has been the subject of domestication). This has enabled the formation of a group of animals which has acquired stable, generally inheritable characteristics”.

Companion animal. A companion animal is an animal receiving protection from humans in exchange for its presence, its beauty, its joviality or also for its talents (songbirds, talking birds . . . ). Owing to their very long presence alongside humankind, these familiar animals have often been the subject of domestication after they have been tamed. However, they are distinguished from domestic animals simply living in the vicinity of the home, a mere commensal to human beings, such as working dogs, and in contrast to what are known as “production animals” which are used for their meat, their milk or their eggs, such as cows or chickens. In western countries, the main companion animals are cats and dogs which, along with the ferret, are animals which are classed as “domestic carnivores”.

New companion animals (NCA). New companion animals (more generally named by the acronym NCA) are companion animals who belong to species other than dogs and/or cats. Animals which can be considered to be NCAs, for example, are ferrets, rabbits, birds, rodents, fish, reptiles, amphibians, insects and spiders, even pigs, fennecs or monkeys which people keep like animals kept for pleasure, with the specific aim of making companion animals thereof.

Fear. As indicated in the preamble of the present patent application, fear is considered to be a state of alert and agitation caused by a present or threatening danger (Sherman and Mills, 2008).

Anxiety. Here again, as indicated in the preamble of the present patent application, anxiety is a response to a possible or imagined danger or to uncertainty (Sherman and Mills, 2008).

Behaviour disorder (also called behavioural disorder). Behaviour disorders are abnormalities in the manner of acting and reacting. The present invention aims in particular to prevent, regulate and/or treat the following behaviour disorders: the act of attacking, aggressiveness, destroying, inappropriate elimination, the act of repeatedly licking a part of its body, the act of scratching itself, astasia (being unable to remain standing), the act of trembling, the act of marking its territory, aberrant motor behaviour, abnormal feeding behaviour such as polyphagia (excessive need to eat, which is not limited by the feeling of satiety) or polydipsia (feeling of excessive thirst despite an excessive consumption of liquid), dysbasia (difficulty in executing movements necessary for walking), an abnormal sensory profile, an abnormal posture, an abnormal vocalisation, sleep disorders, loss of expression, loss of sociability and abnormal judgement of situations. Behaviour disorders in an animal can prove to be extremely problematic, to the extent that these disorders can result in irreversible reactions ranging from abandonment to euthanasia of the animal concerned. Moreover, of course, these behaviour disorders harm the well-being and equilibrium of the animal concerned.

Compound feed for animals. The present invention also relates to a compound feed for animals comprising the veterinary composition according to the invention. In accordance with the regulation (EC n° 767/2009) of the European Parliament and of the Council of 13 Jul. 2009, a “compound feed for animals” denotes a mixture of at least two raw materials for animal feeds, comprising or not comprising additives for feeding animals, which is intended for animal feeding orally, in the form:
i) of a complete feed for animals, or
ii) of a supplementary feed for animals.

Complete feed for animals. The invention also relates to a “complete feed for animals” which comprises the veterinary composition according to the invention as such, defined by the abovementioned regulation EC n° 767/2009 as a compound feed for animals which, owing to its composition, is sufficient to provide a daily ration.

The complete feed for animals comprising the veterinary composition according to the invention can also be considered to be a “feed for animals aimed at specific nutritional objectives”.

Supplementary feed for animals. The present invention also relates to a veterinary nutritional composition or a supplementary feed for animals comprising the veterinary composition according to the invention. As indicated in the regulation (EC n° 767/2009) of the European Parliament and of the Council of 13 Jul. 2009, “supplementary feed for animals” is understood to mean a compound feed for animals which has a high content of certain substances but which, owing to its composition, only provides the daily ration if it is combined with other feeds for animals. The supplementary feed for animals comprising the veterinary composition according to the invention can also be considered to be a “feed for animals aimed at specific nutritional objectives”.

Feed for animals aimed at specific nutritional objectives Feed for animals which is capable of achieving a specific nutritional objective due to its specific composition or its specific manufacturing method, which distinguishes it clearly from ordinary feeds for animals. It can be a complete feed or a supplementary feed.

Specific nutritional objectives. Still according to the abovementioned regulation EC No. 767/2009, the term “specific nutritional objective” denotes an objective which consists in satisfying the specific nutritional needs of animals whose uptake process, absorption process or metabolism is or risks being temporarily or irreversibly disturbed and which, as a result, can draw benefits from the ingestion of feeds for animals which are appropriate for their condition.

The invention also relates to a veterinary functional feed comprising the veterinary composition according to the invention.

### DETAILED DESCRIPTION

The examples below will enable the present invention to be better understood. These examples are given by way of illustration and must under no circumstances be regarded as limiting the scope of said invention in any way.

### Example 1—Method of Preparing Capsules Comprising the Veterinary Composition According to the Invention

The preparation method described hereinafter relates to a production of 1,470 pill tubs of 60 capsules each.

### A. RAW MATERIALS

A.1 Active ingredients

The active ingredients which go into the composition according to the invention are the following:
GABOLYSAT®PTP 55 (preparation based on fish protein hydrolysate)
SOD B Primo-antioxidant® M (5 IU/mg) (melon juice concentrate freeze-dried and coated with palm oil)
A.2 Excipients

The raw materials used as excipients in the composition according to the invention are the following:
MICROCRYSTALLINE CELLULOSE PDR—E460
MAGNESIUM STEARATE—E470b
SILICON DIOXIDE—E551
WHITE GELATIN CAPSULE T100

These materials are stored at a positive temperature.

### B. MIXTURE

The raw materials presented in point A above are weighed individually then mixed in accordance with any suitable method known to the person skilled in the art. If necessary, a raw material can be milled and/or sieved prior to mixing.

Prior to mixing, the manufacture of 1,470 pill tubs requires 59.413 kg of mixed powder, according to the following dosage:
Microcrystalline cellulose (powder)—E460: 8.0685 kg
Silicon dioxide—E551: 0.2475 kg
SOD B Primo-antioxidant® M (5 IU/mg): 1.089 kg
GABOLYSAT®PTP 55: 49.50 kg
Magnesium stearate—E470b: 0.495 kg

The final mixture is bagged, then stored at a positive temperature.

### C. MANUFACTURE OF THE CAPSULES

The capsules used are 720 mg capsules (reference: milgel 4282).

The dosage per capsule is the following:
WHITE GELATIN CAPSULE TOO: weight 120 mg
Microcrystalline cellulose (powder)—E460: 81.50 mg
Silicon dioxide—E551: 2.50 mg
SOD B Primo-antioxidant® M (5 IU/mg): 11 mg
GABOLYSAT®PTP 55: 500 mg
Magnesium stearate—E470b: 5 mg

A sampling of capsules is undertaken by the quality department. Analyses are undertaken according to the internal inspection plan.

Weighings are undertaken throughout the manufacturing process to inspect the weight of the capsules.

The loose capsules are placed in bags, then into barrels for storage at a positive temperature.

### D. PACKAGING/COUNTING

The loose capsules are packaged in 60-capsule pill tubs in accordance with the work instruction associated with the post. The packaging products are the following:
White 125 ml HDPE pill tub
Tamper-proof cap EP43

Once the packaging has been completed, the pill tubs are counted, then placed in boxes for storage at a positive temperature.

### Example 2—Veterinary Clinical Study Performed on Thirty-Nine Dogs

### A. Materials and Methods

A.1 Place

The study was approved by the committee for the use and care of animals at the École nationale vétérinaire d'Alfort et Université Paris-Est [Alfort National Veterinary School and University of Paris-East] (ComERC ENVA; approval number COMERC 2016-01-15). All the dog owners gave their informed consent by way of a written document signed prior to any study procedure. The study took place in the experiment room of the IRCA (Institut de Recherche Clinique Animale [Institute for Animal Clinical Research]) within Alfort National Veterinary School. The 15 m² room was adapted for the behavioural tests and the areas used were carefully marked. The floor area was divided into 1 m² squares.
A.2 Animals
   A.2.1 General inclusion criteria

Companion dogs of various breeds, aged from 1 to 6 years old, were recruited in the waiting room for vaccinations or via the internet. Prior to their inclusion, the dogs were subjected to a general clinical examination in order to check that they were in good health. Aggressive dogs or dogs which were difficult to control were excluded from the study. A check was performed (clicker test with the Clix® multi-clicker) to ensure that all the dogs included had good auditory capacity. Pregnant or lactating dogs and dogs which were on corticosteroids or undergoing psychological treatment were not included. A total of 39 dogs with an average age of 4.0±1.7 years participated in this study. 26 were female and 13 were male (cf. table 7 below).

TABLE 7
Individuals tested

| INCLUSION | | AGE | | CBARQ on | Placebo/Supplement |
|---|---|---|---|---|---|
| NUMBER | SPECIES | (years) | GENDER | day 0 | (S) |
| 160426-ALEFI | AUSTRALIAN SHEPHERD | 6 | f | 1 | P |
| 160426-ALEIG | CHIHUAHUA | 3 | m | 9 | P |
| 160426-ALELI | LABRADOR | 6 | f | 2 | S |
| 160429-CROHU | GERMAN SHEPHERD | 4 | f | 1 | S |
| 160429-ETHIN | CROSS-BREED | 1 | f | 9 | S |
| 160429-HGUNI | CROSS-BREED | 5 | f | 6 | P |
| 160502-MMUEL | CROSS-BREED | 2 | f | 7 | S |
| 160503-CJAEL | CROSS-BREED | 3 | m | 4 | P |
| 160503-MRIKA | CROSS-BREED | 2 | m | 3 | P |
| 160503-MRITI | CROSS-BREED | 4 | m | 3 | S |
| 160510-EBOGE | LABRADOR | 5 | m | 4 | S |
| 160510-EBOJO | BEAGLE | 2 | m | 9 | P |
| 160511-ADEFA | YORKSHIRE TERRIER | 6 | f | 3 | P |
| 160511-ADEKA | YORKSHIRE TERRIER | 4 | f | 2 | S |
| 160513-CGAFA | PYRENEAN SHEPHERD | 6 | f | 8 | P |
| 160516-AGODJ | AUSTRALIAN SHEPHERD | 6 | m | 9 | P |
| 160516-AGOLI | WHIPPET | 5 | f | 1 | P |
| 160516-CAPFL | AUSTRALIAN SHEPHERD | 6 | m | 3 | S |
| 160520-AGOKE | AUSTRALIAN SHEPHERD | 6 | f | 2 | P |
| 160520-AGOTI | BRAZILIAN TERRIER | 2 | f | 2 | P |
| 160520-IBALA | GOLDEN RETRIEVER | 5 | f | 8 | S |
| 160520-MYALE | POODLE | 2 | f | 5 | P |
| 160520-VPIJA | JACK RUSSELL | 5 | m | 8 | P |
| 160520-VPIMO | JACK RUSSELL | 2 | f | 3 | S |
| 160524-SWENI | LABRADOR | 6 | f | 3 | S |
| 160525-CLEFI | GERMAN SHEPHERD | 6 | m | 1 | P |
| 160526-CLEIO | GOLDEN RETRIEVER | 3 | f | 1 | S |
| 160601-HBUNA | SHIBA INU | 3 | f | 7 | S |
| 160607-IBEIY | AKITA | 2 | f | 3 | P |
| 160607-NDAFA | WEST HIGHLAND TERRIER | 5 | f | 8 | S |
| 160607-SJOZA | JACK RUSSELL | 3 | f | 6 | P |
| 160608-CBEEL | BICHON | 6 | m | 8 | P |
| 160608-MLELE | CROSS-BREED | 1 | f | 7 | P |
| 160608-MLEWI | CROSS-BREED | 5 | f | 7 | S |
| 160613-ECOIZ | AUSTRALIAN SHEPHERD | 3 | f | 8 | P |
| 160613-ESELY | CROSS-BREED | 6 | f | 8 | S |
| 160614-ELEHA | CANE CORSO | 4 | f | 6 | S |
| 160614-ELEJA | CANE CORSO | 2 | m | 1 | S |
| 160614-LALPA | CROSS-BREED | 1 | m | 2 | P |
| | AVERAGE VALUE ± SD | 4.0 ± 1.7 | | 4.85 ± 2.86 | |
| | (Standard-Deviation) | | | | |

A.2.2. Inclusion criteria based on a result of behaviour associated with fear which are adapted from the CBARQ (Serpell & Hsu, 2003)

Behavioural evaluations of the dogs were obtained by using a simplified version of the CBARQ (Serpell & Hsu, 2003; Bourienne, 2015), a standardised monitoring instrument having established characteristics of reliability and validity. Four questions from the original questionnaire were selected so that the owners gave a mark corresponding to their own assessment of fear in their dog. The marks for the 4 questions (from 0 to 3 points) were added up, and ranged from 0 to 12. This mark was used as the inclusion criterion: the dogs presenting a result ranging from 1 to 9 were selected for this study. The four questions hereinbelow were marked (cf. table 8 below).

TABLE 8
Simplified CBARQ questionnaire used in order to select the dogs

1. In the presence of loud noises (i.e. cars, fireworks, thunder), does your dog start to tremble,
bark or does he/she try to run away?
□No, never (0 pt) □Yes, sometimes (1 pt) □Yes, frequently (2 pts) □Yes, always (3 pts)
2. When someone unknown to your dog approaches him/her at home, does your dog start to
tremble, bark or does he/she try to run away?
□No, never (0 pt) □Yes, sometimes (1 pt) □Yes, frequently (2 pts) □Yes, always (3 pts)
3. When someone unknown to your dog approaches him/her during a walk, does your dog
start to tremble, bark or does he/she try to run away?
□No, never (0 pt) □Yes, sometimes (1 pt) □Yes, frequently (2 pts) □Yes, always (3 pts)
4. When your dog is left alone at home, does your dog start to bark, destroy furniture,
objects or other things?
□No, never (0 pt) □Yes, sometimes (1 pt) □Yes, frequently (2 pts) □Yes, always (3 pts)

A.3 Experimental protocol
   A.3.1. Tested product (S) and placebo (P)

The tested product is a supplementary feed (abbreviated to “supplement”) presented in the form of a capsule containing 500 mg of a preparation based on a fish protein hydrolysate (GABOLYSAT®PTP 55) and 11 mg of SOD B Primo-antioxidant® M (5 IU/mg). The principal excipient of this feed supplement is microcrystalline cellulose, said feed supplement containing 81.5 mg thereof. The method for preparing the tested product (S) is that presented in example 1 above.

The placebo is a capsule of the same size as that containing the supplement, comprising 445 mg per microcrystalline cellulose capsule, and obtained by adapting the preparation method presented in example 1 above.

The supplement/placebo was given to the dog daily throughout the study (namely for 30 days), from the day after the first test. The supplement was given to 18 dogs and the placebo to 21 dogs.
A.3.2. Protocol

The present test was adapted from a test developed and applied with the aim of observing the behavioural reactions in dogs when faced with various specific situations (Hoummady et al., 2016).

Prior to entering the room, the owner of the dog was asked to fill in the simplified CBARQ questionnaire (cf. table 8 above). A saliva sample was collected with the aid of an oral swab (Salimetrics Kit®) just prior to entering the test room, and just after leaving the room. The tubes containing the saliva were stored at 4° C., then brought to the laboratory in order to be centrifuged there, frozen to −20° C. and then analysed.

The dogs were tested 3 times during a period of 30 days (on day 0—on day 15—on day 30).
A.3.3. Behavioural tests

The dog enters the room without a lead, without its master and the door is closed by an assistant of the experimenter. The test lasted 6 minutes and 50 seconds in all and was divided into 4 sub-tests. The test was recorded using a CANON (EOS 700D) camera, and the videos were then analysed. The experimenter recorded the duration of each sub-test using a stopwatch.

Sub-test 1 (ST1). Exploration of a new environment (3 minutes). The experimenter remains seated on a chair within the experiment area (E) without any physical or visual contact. The dog is left free to explore the layout of the room.

Sub-test 2 (ST2). Interaction with an unknown person (2 minutes). The experimenter stands up from the chair, fetches 2 balls and a string and remains in a low position in the play area (P). He/she calls the dog and invites it to play 3 times.

Sub-test 3 (ST3). Loud noise (1 minute 20 seconds). The experimenter makes a sudden noise which lasts for 20 seconds. This corresponds to a vacuum-cleaner noise of 85 dB (CD Clix®). A conventional CD player was used and a calibration was performed in an acoustic field with the aid of a precision sonometer (type 2235 sonometer with a type 2235 microphone type 1626 +1/3-1/1 Octave Filter Set, Brüel &Kjær Sound & Vibration Measurement A/S, Nærum, Denmark). After having made the noise, the experimenter remains seated for one minute.

Sub-test 4 (ST4). Reaction in relation to an unknown object (30 seconds). From a chair, the experimenter directs a remote-controlled car and places it in area X. The dog is observed throughout this time.

The dogs were tested on day 0, on day 15 and on day 30. For sub-test 2, the experimenter was changed in each session so as to avoid the dog becoming used to the presence of the experimenter. For sub-test 4, the same unknown object was used all three times (the remote-controlled car), so that the dog could become used to the object.

Once the test was finished, the door was opened and the dog was invited to leave the room. The camera was switched off. The videos were then analysed with the BORIS v 2.97 software (Behavioural Observation Research Interactive Software). The behaviours listed in table 9 were observed and quantified by two experimenters whose correlation was tested beforehand with the aid of Kendall's tau coefficient (0.983±0.012 for 11 videos).

TABLE 9
Behavioural results during the test

| Behaviour | Event (E)/State (S) | Description | Sub-test(s) |
|---|---|---|---|
| Interaction with the | E | Bites, claws, touches the door or | ST1, ST2, ST3, ST4 |
| door | | jumps in the direction of the | |
| | | door for at least 2 seconds | |
| | | (one interaction representing 2 | |
| | | seconds) | |
| Door area | S | Time spent in front of the door, | ST1, ST2, ST3, ST4 |
| | | at least half of the body in the | |
| | | door area (no movement for 2 | |
| | | seconds) | |
| Whimpering | E | 1 sound | ST1, ST2, ST3, ST4 |
| Yawning | E | Involuntary opening of the | ST1, ST2, ST3, ST4 |
| | | mouth with extension of the | |
| | | tongue | |
| Licking its | E | Rapid extension of the tongue | ST1, ST2, ST3, ST4 |
| chops/snout | | which passes over the | |
| | | chops and the snout | |
| Experimenter area | S | Time spent in an area less than | ST1, ST3, ST4 |
| | | 1 metre from the experimenter | |
| Traversings of area | E | Number of areas (1 m²) | ST1 |
| | | traversed by at least half of the | |
| | | body | |
| Play | S | Time spent playing with the | ST2 |
| | | toys and/or the experimenter | |
| Turning the head in | E | The dog turns its head in the | ST3 |
| the direction of the | | direction of the source of the | |
| noise | | noise | |
| Approaches an | E | Approaching movement in the | ST4 |
| unknown object | | direction of an unknown object | |
| | | (remote-controlled car) | |
| Turns head towards | E | The dog turns its head in the | ST4 |
| an unknown object | | direction of the unknown | |
| | | object and remains stationary | |
| Touches the | E | The dog touches the unknown | ST4 |
| unknown object | | object (1 contact representing 2 | |
| | | seconds) | |

A.3.4. Dosage of salival cortisol (prior to the sub-tests)

A minimum volume of collected saliva was necessary (50 μl) in order to be able to perform an analysis. A sample was taken for all the dogs, but only 31 of them produced enough saliva. The cortisol was analysed by a specialist laboratory located in the town of Namur (UNamur—Unité de recherche vétérinaire intégrée URVI [Integrated Veterinary Research Unit IVRU]).
A.4 Statistical analyses

Comparisons between the dogs which obtained CBARQs ranging from 1 to 5 and the dogs which obtained CBARQs ranging from 6 to 9 were performed using the Mann-Whitney non-parametric tests.

The comparisons of the behaviours throughout the 3 sessions were performed with the aid of a non-parametric variance analysis on repeated measurements (when normality had not been ascertained).

The comparison of the behaviours and of the deltas (day 15—day 0; day 30—day 0) for the supplement and the placebo was performed using the Mann-Whitney non-parametric tests.

The averages are expressed with ±SD.

### B. Results

B.1 Assessment of the behavioural tests and of the simplified CBARQ questionnaire in relation to fear

The average simplified CBARQ result for the 39 dogs on day 0 was 4.85±2.86 (cf. table 7 above).

In order to confirm that the simplified CBARQ questionnaire can be used as a reliable tool making it possible to categorise and include dogs which present different levels of fear and anxiety, the individuals were divided into two groups according to their result: CBARQ ranging from 1 to 5 (“less fearful” dogs, n=21) and CBARQ ranging from 6 to 9 (“more fearful” dogs, n=18). The two groups were compared from the point of view of cortisol level prior to the test on day 0, and the behaviours were assessed at the time of the test on day 0. The results showed a significant difference in the cortisol level between the two groups on day 0 (p=0.013; U=56; df=30), the dogs having results between 6 and 9 presenting higher values (6.64±3.66 nmol/L as opposed to 5.23±3.24 nmol/L).

As regards the behaviour, six behavioural units showed significant differences between the two groups of dogs (cf. table 10 below): traversing of area during sub-test 1 (ST1) (p=0.007; U=239; df=38), licking of the chops during sub-test 1 (ST1) (p=0.042; U=267; df=38), time spent in the area of the door during sub-test 2 (ST2) (p=0.019; U=409; df=38), play time during sub-test ST2 (p=0.046; U=264; df=38), licking of the chops during sub-test 3 (ST3) (p=0.025; U=262; df=38) and approaching the unknown object during sub-test 4 (ST4) (p=0.023; U=259; df=38).

These results confirm the consistency between the simplified CBARQ result attributed to the dogs and the measurements of stress and the behaviours associated with stress which were observed during the tests.

The most fearful dogs were characterised by a higher cortisol level, lower activity, more time spent in the area of the door, less time spent playing, a lower number of approaches towards the unknown object, a tendency to yawn more, but less licking of the chops.

TABLE 10
Comparison of the cortisol and behaviours of the dogs with
the simplified CBARQ results ranging from 1 to 5 (“less
fearful” dogs) and from 6 to 9 (“more fearful” dogs) (on day 0)

| | Median | CI [25-75] | Median | CI [25-75] | Test | p-Value |
|---|---|---|---|---|---|---|
| | “Less fearful” dogs | | “More fearful” dogs | | | |
| | n = 21 | | n = 18 | | | |
| ST1 Door area | 65 | 26-117 | 75 | 47-122 | 355.5 | 0.490 |
| ST1 Experimenter | 4.5 | 0-26 | 0 | 0-36 | 315 | 0.606 |
| area | | | | | | |
| ST1 Interaction with | 6 | 3.5-13.5 | 8 | 6-12.5 | 349.5 | 0.606 |
| the door | | | | | | |
| ST1 Traversings | 34 | 26.5-43.5 | 17 | 5-30 | 239 | 0.007 |
| between areas | | | | | | |
| ST1 Whimpering | 8 | 0.5-23.5 | 0 | 0-39 | 290 | 0.217 |
| ST1 Licking | 1 | 0-4 | 0 | 0-1 | 267 | 0.042 |
| ST2 Door area | 0 | 0-45 | 33 | 4-82 | 409 | 0.019 |
| ST2 Play | 68 | 0-117 | 7 | 0-27 | 264 | 0.046 |
| ST2 Interaction with | 0 | 0-2 | 1 | 0-6 | 379.5 | 0.139 |
| the door | | | | | | |
| ST2 Whimpering | 0 | 0-9.5 | 0 | 0-7 | 307.5 | 0.415 |
| ST2 Yawning | 0 | 0-0 | 0 | 0-0 | 333.5 | 0.909 |
| ST2 Licking | 0 | 0-3 | 0 | 0-1 | 301.5 | 0.325 |
| ST3 Door area | 14 | 0-66 | 47 | 15-74 | 397.5 | 0.053 |
| ST3 Experimenter area | 0 | 0-13 | 0 | 0-14 | 338.5 | 0.817 |
| ST3 Interaction with | 1 | 0-3.5 | 1 | 0-3.5 | 328.5 | 0.939 |
| the door | | | | | | |
| ST3 Turns head | 1 | 1-2 | 2 | 1-3 | 381 | 0.125 |
| ST3 Yawning | 0 | 0-0 | 0 | 0-9 | 364 | 0.483 |
| ST3 Licking | 1 | 0-2 | 0 | 0-0.5 | 262 | 0.025 |
| ST4 Door area | 0 | 0-10 | 9 | 0-28 | 382 | 0.117 |
| ST4 Experimenter area | 0 | 0-0 | 0 | 0-6 | 353 | 0.362 |
| ST4 Interaction with | 0 | 0-0 | 0 | 0-0.5 | 336 | 0.869 |
| the door | | | | | | |
| ST4 Turns head | 2 | 1-4.5 | 2 | 0.5-4 | 310 | 0.528 |
| ST4 Approaches object | 1 | 0.5-1 | 0 | 0-1 | 259 | 0.023 |
| ST4 Touches object | 0 | 0-1 | 0 | 0-0.5 | 310.5 | 0.463 |
| ST4 Whimpering | 0 | 0-0.5 | 0 | 0-1 | 342.5 | 0.662 |
| ST4 Yawning | 0 | 0-0 | 0 | 0-0 | 314 | 0.211 |
| ST4 Licking | 0 | 0-0.5 | 0 | 0-0 | 309 | 0.337 |
| | “Less fearful” dogs | | “More fearful” dogs | | | |
| | n = 17 | | n = 14 | | | |
| Cortisol prior to the | 3.139 | 2.655-4.798 | 5.694 | 3.972-8.188 | 287 | 287 |
| sub-tests in nmol/L | | | | | | |

Once the information on the placebo and the supplement has been revealed, the averages of the simplified CBARQ results on day 0 were calculated. The median results for the placebo were 5.0 (QI ranging from [2.0; 8.0]) and the median results for the supplement were 3.5 (QI ranging from [2.75; 7.25]). There was no significant difference between the two groups of dogs in terms of level of fear (U=176, p=0.722, df=38). The dogs had therefore been affected in an equal way in the group which received the placebo or the group which received the supplement. This means that the two groups of dogs (“less fearful dogs” and “more fearful dogs” are homogenous).
B.2 Comparison between the placebo and the supplement: behavioural tests
   B.2.1 Comparison on day 0

On day 0, no difference was detected between the group which received the placebo and the group which received the supplement (cf. table 11 below). However, the dogs of the group which received the placebo showed a tendency to be more active during sub-test 1 (p=0.055) and to remain in the area of the door during sub-test 2 (p=0.09).

TABLE 11
Comparisons of placebo and supplement on the
behaviours during the different sub-tests

| | Day 0 | | | | | |
|---|---|---|---|---|---|---|
| | Supplement N = 18 | | Placebo N = 21 | | | |
| ST Behaviour | Median | CI [25-75] | Median | CI [25-75] | Test | p-Value |
| ST1 Door area | 50.9 | 21.1-132.7 | 74.9 | 54.1-118.1 | 336 | 0.508 |
| ST1 Experimenter area | 2.9 | 0-29.8 | 0 | 0-20.4 | 382.5 | 0.494 |
| ST1 Interaction with | 6.5 | 4.5-11.5 | 8 | 4-16 | 338.5 | 0.553 |
| the door | | | | | | |
| ST1 Traversing | 22.5 | 9.7-33.2 | 33 | 24.5-47.5 | 291.5 | 0.055 |
| ST1 Whimpering | 6 | 0-22.7 | 4 | 0-46 | 342 | 0.614 |
| ST1 Yawning | 0 | 0-0 | 0 | 0-0 | 373 | 0.544 |
| ST1 Licking | 1 | 0-2.2 | 0 | 0-2.5 | 386.5 | 0.424 |
| ST2 Door area | 0 | 0-32 | 43.2 | 0-60.8 | 302 | 0.09 |
| ST2 Play | 27 | 0-117.3 | 10.9 | 0-84.9 | 401 | 0.245 |
| ST2 Interaction with | 0 | 0-2.2 | 1 | 0-6.5 | 317.5 | 0.212 |
| the door | | | | | | |
| ST2 Whimpering | 0 | 0-0.2 | 0 | 0-14 | 319 | 0.184 |
| ST2 Yawning | 0 | 0-0 | 0 | 0-0 | 342 | 0.197 |
| ST2 Licking | 0 | 0-2 | 0 | 0-1.5 | 358.5 | 0.974 |
| ST3 Door area | 17.9 | 2.5-72.3 | 28.4 | 2.1-67 | 346.5 | 0.906 |
| ST3 Experimenter area | 0 | 0-21.1 | 0 | 0-5.2 | 374.5 | 0.415 |
| ST3 Interaction with | 1 | 0-4.2 | 1 | 0-2.7 | 363.5 | 0.717 |
| the door | | | | | | |
| ST3 Turns head in the | 1 | 1-3 | 2 | 1-3 | 317.5 | 0.304 |
| direction | | | | | | |
| ST3 Whimpering | 2 | 0-10.7 | 2 | 0-5.5 | 371 | 0.552 |
| ST3 Yawning | 0 | 0-0 | 0 | 0-0 | 325 | 0.203 |
| ST3 Licking | 0.5 | 0-2 | 0 | 0-1 | 375.5 | 0.439 |
| ST4 Door area | 5.1 | 0-26.7 | 0 | 0-17 | 372.5 | 0.512 |
| ST4 Experimenter | 0 | 0-1.2 | 0 | 0-0 | 362 | 0.650 |
| area | | | | | | |
| ST4 Interaction with | 0 | 0-1 | 0 | 0-0 | 374.5 | 0.345 |
| the door | | | | | | |
| ST4 Turns head in | 1 | 1-4 | 2 | 1-4.7 | 332.5 | 0.59 |
| the direction | | | | | | |
| ST4 Approaches object | 1 | 0-1.2 | 0.5 | 0-1 | 370.5 | 0.549 |
| ST4 Touches object | 0 | 0-1 | 0 | 0-1 | 351 | 1 |
| ST4 Whimpering | 0 | 0-1 | 0 | 0-0 | 373.5 | 0.406 |
| ST4 Yawning | 0 | 0-0 | 0 | 0-0 | 371 | 0.141 |
| ST4 Licking | 0 | 0-1 | 0 | 0-0 | 380.5 | 0.208 |

B.2.2. Comparison on day 15

On day 15, the dogs of the group which received the supplement had a tendency to remain fora longer time in the experimenter area during sub-test 1 (p=0.061; cf. table 12 below).

TABLE 12
Comparisons of placebo and supplement behaviours during the different sub-tests

| | Day 15 | | | | | |
|---|---|---|---|---|---|---|
| | Supplement N = 18 | | Placebo N = 21 | | | |
| ST Behaviour | Median | CI [25-75] | Median | CI [25-75] | Test | p-Value |
| ST1 Door area | 70.9 | 23-126.6 | 102.4 | 53.7-142.6 | 324 | 0.438 |
| ST1 Experimenter area | 18.5 | 0-70.6 | 1.9 | 0-23.1 | 413.5 | 0.061 |
| ST1 Interaction with | 5 | 2.7-8 | 5.5 | 3-14.5 | 333 | 0.607 |
| the door | | | | | | |
| ST1 Whimpering | 9 | 1.5-21 | 9 | 0-36.7 | 356 | 0.894 |
| ST1 Yawning | 0 | 0-0 | 0 | 0-0 | 381 | 0.246 |
| ST1 Licking | 1.5 | 0-4 | 1 | 0-3 | 382 | 0.352 |
| ST2 Door area | 20 | 0-71.6 | 22.7 | 0-64.7 | 342.5 | 0.812 |
| ST2 Play | 9.4 | 0-90.5 | 2.9 | 0-80.8 | 377 | 0.442 |
| ST2 Interaction with | 1 | 0-4 | 1 | 0-5 | 339 | 0.726 |
| the door | | | | | | |
| ST2 Whimpering | 0 | 0-8 | 0 | 0-20.5 | 345 | 0.858 |
| ST2 Yawning | 0 | 0-0 | 0 | 0-0 | 352 | 0.978 |
| ST2 Licking | 2 | 0-3.250 | 1 | 0-2.7 | 373 | 0.517 |
| ST3 Door area | 26.7 | 0-61.5 | 25.5 | 12.6-62.6 | 343 | 0.825 |
| ST3 Experimenter area | 0 | 0-15 | 0 | 0-29.2 | 357 | 0.855 |
| ST3 Interaction with | 1.5 | 0-5.2 | 0.5 | 0-2.7 | 381.5 | 0.36 |
| the door | | | | | | |
| ST3 Turns head in the | 1 | 1-1.2 | 1 | 1-2 | 356 | 0.881 |
| direction | | | | | | |
| ST3 Whimpering | 1.5 | 0-6.2 | 0 | 0-17 | 351.5 | 1 |
| ST3 Yawning | 0 | 0-0.2 | 0 | 0-1 | 331 | 0.459 |
| ST3 Licking | 1 | 0-2.2 | 0.5 | 0-1.7 | 383 | 0.331 |
| ST4 Door area | 10.9 | 0-27.9 | 5.3 | 0-25.3 | 372 | 0.539 |
| ST4 Experimenter area | 0 | 0-2 | 0 | 0-5.2 | 341 | 0.72 |
| ST4 Interaction with | 0 | 0-0.25 | 0 | 0-0.7 | 346 | 0.86 |
| the door | | | | | | |
| ST4 Turns head in the | 1 | 1-3.5 | 2 | 1-4.5 | 336 | 0.656 |
| direction | | | | | | |
| ST4 Approaches object | 0 | 0-1.2 | 0 | 0-1 | 370.5 | 0.519 |
| ST4 Touches object | 0 | 0-25 | 0 | 0-0 | 360.5 | 0.697 |
| ST4 Whimpering | 0 | 0-1 | 0 | 0-3.5 | 346 | 0.873 |
| ST4 Yawning | 0 | 0-0 | 0 | 0-0 | 361 | 0.317 |
| ST4 Licking | 0 | 0-1 | 0 | 0-0 | 383.5 | 0.241 |

B2.3. Comparison on day 30

On day 30, the dogs of the group which received the supplement were less prone to yawning during sub-test 2 (p=0.057). No dog from the group which received the supplement yawned (0/18), whereas three dogs from the group which received the placebo (3/21) yawned during sub-test 2 (cf. table 13 below).

TABLE 13
Placebo and supplement comparisons of the behaviours during the different sub-tests

| | Day 30 | | | | | |
|---|---|---|---|---|---|---|
| | Supplement N = 18 | | Placebo N = 21 | | | |
| ST Behaviour | Median | CI [25-75] | Median | CI [25-75] | Test | p-Value |
| ST1 Door area | 79.5 | 15-143 | 67 | 42.7-125.4 | 357 | 0.944 |
| ST1 Experimenter area | 10 | 0-50.1 | 0 | 0-37.8 | 394.5 | 0.321 |
| ST1 Interaction with | 6 | 1-10.5 | 7 | 3-10.5 | 342 | 0.621 |
| the door | | | | | | |
| ST1 Traversings | 18.5 | 12.7-37.7 | 25 | 13.5-40.5 | 352.5 | 0.844 |
| ST1 Whimpering | 10.5 | 3-37.5 | 18 | 0-34.5 | 366.5 | 0.865 |
| ST1 Yawning | 0 | 0-0 | 0 | 0-0.5 | 324.5 | 0.116 |
| ST1 Licking | 1 | 0-4 | 1 | 0-2 | 374 | 0.688 |
| ST2 Door area | 4.6 | 0-100.5 | 33 | 2.5-71 | 344.5 | 0.665 |
| ST2 Play | 23.9 | 0-96 | 11 | 0-46.7 | 381 | 0.558 |
| ST2 Interaction with | 0.5 | 0-3.2 | 4 | 0-8.5 | 324.5 | 0.294 |
| the door | | | | | | |
| ST2 Whimpering | 0 | 0-2.7 | 0 | 0-16 | 312.5 | 0.131 |
| ST2 Yawning | 0 | 0-0 | 0-0 | 0-0 | 324 | 0.057 |
| ST2 Licking | 0 | 0-0.5 | 0 | 0-2.5 | 322.5 | 0.213 |
| ST3 Door area | 49.8 | 5.15-66.7 | 42 | 9-77.75 | 345 | 0.682 |
| ST3 Experimenter area | 0 | 0-11.4 | 0 | 0-34.2 | 329 | 0.317 |
| ST3 Interaction with | 2 | 0-4.2 | 1 | 0-4 | 375 | 0.677 |
| the door | | | | | | |
| ST3 Turns head in the | 2 | 1-3 | 2 | 1-3 | 379.5 | 0.581 |
| direction | | | | | | |
| ST3 Whimpering | 2.5 | 0-7.5 | 3 | 0-15 | 365 | 0.895 |
| ST3 Yawning | 0 | 0-0 | 0 | 0-0 | 358 | 0.950 |
| ST3 Licking | 0.5 | 0-2.2 | 1 | 0-1 | 362 | 0.964 |
| ST4 Door area | 6 | 0-29.2 | 13 | 0-30 | 353 | 0.848 |
| ST4 Experimenter area | 0 | 0-1 | 0 | 0-7.4 | 347 | 0.646 |
| ST4 Interaction with | 0 | 0-2.2 | 0 | 0-0.5 | 382 | 0.445 |
| the door | | | | | | |
| ST4 Turns head in the | 1 | 1-2 | 2 | 1-2.5 | 321 | 0.254 |
| direction | | | | | | |
| ST4 Approaches object | 0 | 0-1 | 0 | 0-0 | 375.5 | 0.565 |
| ST4 Touches object | 0 | 0-0 | 0 | 0-0 | 351 | 0.680 |
| ST4 Whimpering | 0 | 0-4.5 | 0 | 0-2.5 | 376 | 0.611 |
| ST4 Yawning | 0 | 0-0 | 0 | 0-0 | 351 | 0.382 |
| ST4 Licking | 0 | 0-1 | 0 | 0-0.5 | 367.5 | 0.797 |

B.3 Placebo and supplement comparison of the variations between day 0 and day 30: behavioural tests

Between day 30 and day 0, the dogs from the group which received the supplement saw their activity increase whereas the dogs from the group which received the placebo saw their activity decrease (p=0.036) during sub-test 1 (cf. table 14 below).

In addition, the dogs from the group which received the supplement were less prone to yawning between day 0 and day 30 (p=0.086) during sub-test 4: two dogs from the group which received the supplement saw their yawning reduce whereas one dog from the group which received the placebo saw its yawning increase (cf. table 14 below).

TABLE 14
Placebo and supplement comparisons of the variations between
day 30 and day 0 for behaviours of different sub-tests

| | Day 30 as opposed to Day 0 | | | | | |
|---|---|---|---|---|---|---|
| | Supplement N = 18 | | Placebo N = 21 | | | |
| ST Behaviour | Median | CI [25-75] | Median | CI [25-75] | Test | p-Value |
| ST1 Door area | −5.2 | −70.1-64.1 | 5.6 | −25.1-30.6 | 362 | 0.966 |
| ST1 Experimenter area | 7.2 | −10.3-45.7 | 0 | −10.4-18.6 | 386 | 0.467 |
| ST1 Interaction with | −1 | −6.2-5 | 0 | −5.5-3.5 | 340 | 0.582 |
| the door | | | | | | |
| ST1 Traversings | 5.5 | −13-11 | −15 | −20-0.5 | 435 | 0.036 |
| ST1 Whimpering | 5 | 1.7-11 | 0 | −3-10.5 | 402.5 | 0.234 |
| ST1 Yawning | 0 | 0-0 | 0 | 0-0.5 | 317 | 0.105 |
| ST1 Licking | 0 | −1-2.2 | 0 | 0-2 | 364 | 0.919 |
| ST2 Door area | 0 | −4.5-58.7 | 0 | −28.1-37.5 | 376 | 0.66 |
| ST2 Play | 0 | −26.3-4.6 | 0 | −6.2-10.9 | 346 | 0.703 |
| ST2 Interaction with | 0 | −1.2-2.5 | 0 | −1-4.5 | 347 | 0.72 |
| the door | | | | | | |
| ST2 Whimpering | 0 | 0-0.5 | 0 | 0-10.5 | 329.5 | 0.364 |
| ST2 Yawning | 0 | 0-0 | 0 | 0-0 | 342 | 0.432 |
| ST2 Licking | 0 | −1-0 | 0 | −1-0.5 | 343 | 0.612 |
| ST3 Door area | 3.2 | −9.4-24 | 7.3 | −6.8-27.8 | 333 | 0.609 |
| ST3 Experimenter area | 0 | −2.5-0 | 0 | −5.9-34.5 | 321 | 0.362 |
| ST3 Interaction with | 0 | −1-2.5 | 0 | −1-3 | 355 | 0.906 |
| the door | | | | | | |
| ST3 Turns head in the | 0 | −0.2-1.2 | 0 | −2-1.7 | 382.5 | 0.356 |
| direction | | | | | | |
| ST3 Whimpering | 0 | −4.2-2.5 | 0 | −2-5.5 | 328 | 0.505 |
| ST3 Yawning | 0 | 0-0 | 0 | 0-0 | 371 | 0.423 |
| ST3 Licking | 0 | −2-2.2 | 0.5 | −1-1 | 331.5 | 0.573 |
| ST4 Door area | 0 | −0.8-5.3 | 4.2 | 0-16.2 | 305 | 0.177 |
| ST4 Experimenter area | 0 | 0-1 | 0 | 0-0 | 369 | 0.545 |
| ST4 Interaction with | 0 | −0.2-1.5 | 0 | 0-0 | 355.5 | 0.896 |
| the door | | | | | | |
| ST4 Turns head in the | 0 | −2-1 | 0 | −2-1 | 342.5 | 0.812 |
| direction | | | | | | |
| ST4 Approaches object | 0 | −1-0 | 0 | −1-0 | 323.5 | 0.39 |
| ST4 Touches object | 0 | −1-0 | 0 | −0.7-0 | 334.5 | 0.569 |
| ST4 Whimpering | 0 | −0.2-3.2 | 0 | 0-1.5 | 364 | 0.696 |
| ST4 Yawning | 0 | 0-0 | 0 | 0-0 | 323 | 0.086 |
| ST4 Licking | 0 | 0-0 | 0 | 0-0.7 | 323 | 0.329 |

B.4. Placebo and supplement variations in the behaviours of the dogs during the tests between days 0, 15 and 30
   B.4.1. Group which received the supplement

Between day 0, day 15 and day 30, a significant difference was detected between the behaviours of the dogs of the group which received the supplement (cf. table 15 below) as regards the time spent in the experimenter area. Indeed, a significant difference was observed in this regard at day 0, in comparison with day 15 and with day 30; dogs remained for a longer time in the experimenter area on day 15 and on day 30 (p=0.078). This could be explained by a less substantial stress reaction, insofar as the experimenter is an unknown person.

TABLE 15
Group which received the supplement: variations in the
behaviours over the 3 samplings: day 0, day 15, day 30

| SUPPLEMENT N = 18 | | |
|---|---|---|
| ST Behaviour | | p-Value |
| ST1 Door area | X² = 1.000, df = 2 | 0.607 |
| ST1 Experimenter | X² = 5.115, df = 2 | 0.078 |
| area | | |
| ST1 Interaction with | X² = 2.030, df = 2 | 0.362 |
| the door | | |
| ST1 Traversing | X² = 1.853, df = 2 | 0.396 |
| ST1 Whimpering | X² = 2.000, df = 2 | 0.368 |
| ST1 Yawning | X² = 4.455, df = 2 | 0.108 |
| ST1 Licking | X² = 0.295, df = 2 | 0.863 |
| ST2 Door area | X² = 0.520, df = 2 | 0.771 |
| ST2 Play | X² = 1.661, df = 2 | 0.436 |
| ST2 Interaction with | X² = 1.814, df = 2 | 0.404 |
| the door | | |
| ST2 Whimpering | X² = 2.971, df = 2 | 0.226 |
| ST2 Yawning | X² = 4.000, df = 2 | 0.135 |
| ST2 Licking | X² = 9.882, df = 2 | 0.007 |
| ST3 Door area | X² = 0.812, df = 2 | 0.666 |
| ST3 Experimenter | X² = 1.946, df = 2 | 0.378 |
| area | | |
| ST3 Interaction with | X² = 0.286, df = 2 | 0.867 |
| the door | | |
| ST3 Turns head in the | X² = 4.128, df = 2 | 0.127 |
| direction | | |
| ST3 Whimpering | X² = 1.080, df = 2 | 0.583 |
| ST3 Yawning | X² = 2.000, df = 2 | 0.368 |
| ST3 Licking | X² = 1.509, df = 2 | 0.470 |
| ST4 Door area | X² = 1.368, df = 2 | 0.504 |
| ST4 Experimenter | X² = 1.040, df = 2 | 0.595 |
| area | | |
| ST4 Interaction with | X² = 1.389, df = 2 | 0.499 |
| the door | | |
| ST4 Turns head in the | X² = 0.500, df = 2 | 0.779 |
| direction | | |
| ST4 Approaches | X² = 10.759, df = 2 | 0.005 |
| object | | |
| ST4 Touches object | X² = 5.360, df = 2 | 0.069 |
| ST4 Whimpering | X² = 0.974, df = 2 | 0.614 |
| ST4 Yawning | X² = 3.000, df = 2 | 0.233 |
| ST4 Licking | X² = 1.400, df = 2 | 0.497 |

B.4.2. Group which received the placebo

Between day 0, day 15 and day 30, some significant differences were detected between the behaviours of the dogs of the group which received the placebo (cf. table 16 below).

The traversing of area was different from one day to another (p =0.032). In particular, the number of traversings of area has reduced on day 30 compared with day 0, indicating that dogs were less active on day 30. This could indicate that the dogs were more stressed on day 30 (less activity).

The touching of the unknown object (sub-test 4) often presented differences between the three sessions: on day 0, the dogs generally touched the object more than on day 15 and on day 30. This could indicate, in the same way as for the traversing of area, that the dogs were more stressed on day 15 and on day 30.

TABLE 16
Group which received the placebo: variations in the behaviours
over the 3 sampling periods: day 0, day 15, day 30

| PLACEBO N = 21 | | |
|---|---|---|
| ST Behaviour | | p-Value |
| ST1 Door area | X² = 0.1000, df = 2 | 0.951 |
| ST1 Experimenter area | X² = 0.275, df = 2 | 0.872 |
| ST1 Interaction with the | X² = 0.494, df = 2 | 0.781 |
| door | | |
| ST1 Traversing | X² = 6.861, df = 2 | 0.032 |
| ST1 Whimpering | X² = 0.0328, df = 2 | 0.984 |
| ST1 Yawning | X² = 2.000, df = 2 | 0.368 |
| ST1 Licking | X² = 1.480, df = 2 | 0.477 |
| ST2 Door area | X² = 0.375, df = 2 | 0.829 |
| ST2 Play | X² = 1.016, df = 2 | 0.602 |
| ST2 Interaction with the | X² = 0.679, df = 2 | 0.712 |
| door | | |
| ST2 Whimpering | X² = 1.409, df = 2 | 0.494 |
| ST2 Yawning | X² = 1.143, df = 2 | 0.565 |
| ST2 Licking | X² = 1.480, df = 2 | 0.477 |
| ST3 Door area | X² = 2.676, df = 2 | 0.262 |
| ST3 Experimenter area | X² = 1.609, df = 2 | 0.447 |
| ST3 Interaction with the | X² = 1.825, df = 2 | 0.402 |
| door | | |
| ST3 Turns head in the | X² = 8.036, df = 2 | 0.018 |
| direction | | |
| ST3 Whimpering | X² = 0.154, df = 2 | 0.926 |
| ST3 Yawning | X² = 1.357, df = 2 | 0.507 |
| ST3 Licking | X² = 0.931, df = 2 | 0.628 |
| ST4 Door area | X² = 8.654, df = 2 | 0.013 |
| ST4 Experimenter area | X² = 0.389, df = 2 | 0.823 |
| ST4 Interaction with the | X² = 3.130, df = 2 | 0.209 |
| door | | |
| ST4 Turns head in the | X² = 0.406, df = | 0.816 |
| direction | | |
| ST4 Approaches | X² = 3.368, df = 2 | 0.186 |
| object | | |
| ST4 Touches object | X² = 5.083, df = 2 | 0.079 |
| ST4 Whimpering | X² = 0.231, df = 2 | 0.891 |
| ST4 Yawning | X² = 2.000, df = 2 | 0.368 |
| ST4 Licking | X² = 1.040, df = 2 | 0.595 |

df = degree of freedom

### C. Conclusions

C.1. Validation of the behavioural tests

In line with the CBARQ as the inclusion criterion, it was considered, for the purposes of the present study, that when an enquiry by questionnaire was used as a tool making it possible to determine animal emotions such as fear, joy and aggressiveness, its validity is of the greatest importance. An external validity indicates the effectiveness with which the measurement conceptually predicts pertinent behaviours, results or criteria (John and Soto, 2007). In this case, it was validated successfully by discovering significant differences between the “less fearful” and “more fearful” dogs (according to their CBARQ score). As Van den Berg (2010) suggested, this questionnaire can be used in complete confidence as a research tool in order to compare behaviours in different canine populations in this type of study.

By comparing the dogs which have a score between 6 and 9 (“more fearful”) for the CBARQ and the dogs which have a score between 1 and 5 (“less fearful”), the results show that the dogs which were more fearful presented a higher level of cortisol, less activity, more time spent in the area of the door, less time spent playing, and a lower number of approaches towards the unknown object, a tendency to yawn more but more rare licking of the chops.
C.2. Behavioural effects of the supplement on the dogs

Once the information on the placebo and on the supplement was revealed, the averages of the simplified CBARQ scores on day 0 were calculated. There was no significant difference between the two groups of dogs concerning the level of fear, therefore the dogs were equally affected in the placebo and supplement groups.
C.2.1. Comparisons of the behaviours between the placebo and supplement groups

On day 15, the dogs of the group which received the supplement had a tendency to remain for a longer time in the experimenter area during sub-test 1 (p=0.061).

On day 30, the dogs of the group which received the supplement were less prone to yawning during sub-test 2 (p=0.057).

The supplement thus appears to facilitate dog-human interaction and to reduce emotionality during interactions with humans.

Between day 0 and day 30, the dogs of the group which received the supplement were more active, whereas the dogs of the group which received the placebo were less active (p =0.036) during sub-test 1. In addition, the dogs of the group which received the supplement had a tendency to yawn less between day 0 and day 30 (p=0.086) during sub-test 4.

On day 30, the supplement appears to facilitate the learning and familiarisation processes in the dogs, by increasing activity. Indeed, the dogs of the group which received the supplement still explored the environment during the third test session. In addition, the dogs yawned less and consequently showed less emotionality.
C.2.2. Changes in the behaviours over the 30 days for the group which received the placebo and the group which received the supplement

### GROUP WHICH RECEIVED THE SUPPLEMENT

Between day 0, day 15 and day 30, a significant difference was observed on day 0 in comparison with day 15 and day 30; the dogs remained for a longer time in the experimenter area on day 15 and on day 30 (p=0.078). This can presumably be explained by a less substantial stress reaction, insofar as the experimenter is an unknown person. Indeed, during the tests, the person was changed between each session and the dogs therefore could not become familiar with the experimenter.

### GROUP WHICH RECEIVED THE PLACEBO

The traversing of the area during sub-test 1 was different from one day to another (p=0.032). In particular, the number of traversings of area reduced on day 30 in comparison with day 0, which indicates that the dogs were less active on day 30. This could indicate that the dogs were more stressed on day 30 (less activity).

Touching the unknown object (sub-test 4) had a tendency to vary from one session to another: on day 0, the dogs had a tendency to touch the object more than on day 15 and on day 30. This could indicate, in the same way as for the traversing of area, that the dogs were more stressed on day 15 and on day 30.

By comparing the changes for the group which received the supplement and for the group which received the placebo, it appears that the dogs of the group which received the supplement seem to explore more and be more curious than the dogs of the group which received the placebo. These dogs therefore could be more motivated to explore and quicker to assimilate new learning.

### D. General Conclusion

The results of the present veterinary clinical study suggest an increase in activity, familiarity with a new person and curiosity, with a reduction in behaviour associated with stress, fear and/or anxiety for dogs which have received the supplement in comparison with those of the group which received the placebo.

The supplement tested therefore proves to be effective in reducing reactions to stress, fear and/or anxiety (in particular in the context of daily mild stress factors), and promoting learning processes.

Furthermore, the present study has made it possible to prove the existence of positive effects on dog-human communication and on learning processes caused by chronic stress factors.

### BIBLIOGRAPHY

Araujo et al 2010 ANXITANE® Tablets reduce fear of human beings in a laboratory model of anxiety-related behavior journal of veterinary behavior 2010 5, 268-275

Bamberger and Houpt 2006. Signalment factors, co-morbidity, and trends in behavior diagnoses in dogs: 1,644 cases (1991e2001). J. Am. Vet. Med. Assoc. 229, 1591-1601.

Beata et al 2007. Effects of alpha-casozepine (Zylkene)versus selegiline hydrochloride (Selgian, Anipryl) on anxiety disorders in dogs. J. Vet. Behay.: Clin. App. Res. 2, 175-183

Bourrienne M. 2015. Développement chez le chien et problèmes comportementaux. [Development in dogs and behavioural problems] Thèse vétérinaire, Maisons-Alfort, ENVA.

Casey, R., 2002. Fear and stress. In: Horwitz, D., Mills, D., Heath, S. (Eds.), BSAVA Manual of Canine and Feline Behavioral Medicine. BSAVA, Gloucester, UK, pp. 144-153.

Clairbone. Catalase activity. In Handbook of Methods for Oxygen Radical Research; Greewald, R. A., ed.; CRC Press: Boca Raton, Fla., 1985; pp 283-284

Denenberg et Landsberg 2008. Effect of dog-appeasing pheromones on anxiety and fear in puppies during training and on long term socialization. J. Am. Vet. Med. Assoc. 233, 1874-1882

Hoummady S. et al. 2016 Relationships between personality of human—dog dyads and performances in working tasks. Applied Animal Behaviour Science. 177: 42-51.

John & Soto, 2007. The importance of being valid. in Handbook of Research Methods in Personality Psychology edited by Richard W Robins R Chris Fraley Robert F

Kato, M., Miyaji, K., Ohtani, N., Ohta, M., 2012. Effects of prescription diet on dealing with stressful situations and performance of anxiety-related behaviors in privately owned anxious dogs. J. Vet. Behay.: Clin. Appl. Res. 7, 21-26.

Korpivaraa, M. Lappas K., Huhtinen M., Schöning B., Overall K. Dexmedetomidine oromucosal gel for noise-assoxiated acute anxiety and fear in dogs—a randomized, double-blind, placebo-controlled clinical study. Veterinary record Apr. 8, 2017 doi: 10.1136/vr104045

Lalles, J-P. “A melon pulp concentrate rich in superoxide dismutase reduces stress proteins along the gastrointestinal tract of pigs”. 2010. Science Direct.

Landsberg G., Mougeot I, Kelly S. & Milgram N. 2015. Assessment of noise-induced fear and anxiety in dogs: Modification by a novel fish hydrolysate supplemented diet. Journal of Veterinary Behavior: Clinical Applications and Research. 10: 391-398.

Milesi et al 2009 effect of oral supplementation with a proprietary melon juice concentrate (extramel®) on stress and fatigue in healthy people: a pilot, double-blind, placebo-controlled clinical trial. NutrJ. 2009; 8-40

Muth et al 2004 influence of an orally effective SOD on hyperbaric-related cell damage free rad res 38 927-932

Notin C., Vallon L., Desbordes F., Leleu C., 2010. Oral supplementation with superoxide dismutase in Standardbred trotters in training: a double-blind placebo-controlled study. Equine veterinary journal. 42: 375-381.

Oberley; Spitz. Nitroblue tetrazolium. In Handbook of Methods for Oxygen Radical Research; Greenwald, R. A., Ed.; CRC Press: Boca Raton, Fla., 1985; pp 217-220.

Palestrini, C., Minero, M., Cannas, S., Berteselli, G., Scaglia, E., Barbieri, S., Cavallone, E., Puricelli, M., Servida, F., Dall'Ara, P., 2010. Efficacy of a diet containing caseinate hydrolysate on signs of stress in dogs. J. Vet. Behay.: Clin. Appl. Res. 5, 309-317

Serpell, J. A., Hsu Y. 2003. Development and validation of a questionnaire for measuring behavior and temperament traits in pet dogs. Journal of the American Veterinary Medical Association. 223: 1293-1300

Sherman, B. L. & Mills, D. S (2008) Canine anxieties and phobias: an update on separation anxiety and noise aversions. Veterinary clinics of North America: small animal practice 38, 1081-1106

Sileo (dexmedetomidine 0.1 mg/ml oromucosal gel for dogs) [summary of products characterisitics] Orion Corporation(2015) [last uptdated Oct. 10, 2017 cited Nov. 22, 2017]. Retrieved from http://www.ema.europa.eu

Tiira K. & Lohii H. 2014. Reliability and validity of a questionnaire survey in canine research. Applied Animal Behaviour Science.155: 82-92

Van den Berg SM, Heuven HCM, Van den Berg L, et al. 2010. Evaluation of the C-BARQ as a measure of stranger-directed aggression in three common dog breeds. Applied Animal Behaviour Science. 124:136-141

Vouldoukis, I., Lacan, D., Kamate, C., et al. 2004. Antioxidant and anti-inflammatory properties of a Cucumis melo extract rich in superoxide dismutase activity. Journal of ethnopharmacology. 94: 67-75.

1. A veterinary composition, which can preferably be administered orally, comprising, in effective amounts:
superoxide dismutase or at least one source of superoxide dismutase, and
at least one protein hydrolysate or at least one preparation based on at least one protein hydrolysate, said at least one protein hydrolysate advantageously being devoid of proteins, said at least one protein hydrolysate comprising a peptide fraction in which less than 1% by weight of the peptides have a molecular weight greater than or equal to 10,000 Da, preferably in which 100% by weight of the peptides have a molecular weight less than 10,000 Da, preferably less than 3,000 Da, advantageously less than or equal to 1,800 Da, in a preferred manner less than 1,800 Da.

2. The composition according to claim 1, wherein said peptide fraction represents at least 50% by weight, preferably more than 50% by weight, preferably at least 55% by weight, advantageously at least 60% by weight, in a preferred manner more than 60% by weight, relative to the total weight of said at least one protein hydrolysate.

3. The composition according to claim 1 or 2 comprising at least one source of superoxide dismutase and at least one preparation based on at least one protein hydrolysate in a weight ratio between 0.01:100 and 100:1, preferably between 1:100 and 1:10, preferably between 1:100 and 5:100, advantageously between 1:50 and 2:50.

4. The composition according to any one of the preceding claims, wherein said superoxide dismutase is of plant origin, preferably of fruit origin, advantageously derived from Olea europeae, from Vitis vinifera and/or from at least one Cucurbitaceae such as Cucumis melo; in a preferred manner said superoxide dismutase consisting of the superoxide dismutase of Cucumis melo.

5. The composition according to any one of the preceding claims, wherein said at least one protein hydrolysate is an animal protein hydrolysate or a plant protein hydrolysate.

6. The composition according to any one of the preceding claims, wherein said at least one protein hydrolysate is:
a protein hydrolysate of at least one marine animal, preferably a fish protein hydrolysate, advantageously a protein hydrolysate of fish belonging to the Gadidae family, or
a plant protein hydrolysate.

7. The composition according to any one of the preceding claims, wherein said at least one protein hydrolysate is a protein hydrolysate of at least one marine animal, preferably a fish protein hydrolysate, advantageously a protein hydrolysate of fish belonging to the Gadidae family.

8. The composition according to any one of claims 1 to 7, said composition comprising:
a) from 0.5 to 100 IU, preferably from 20 to 80 IU, preferably from 40 to 65 IU, advantageously from 50 to 60 IU, of superoxide dismutase, and/or
b) from 10 to 1,000 mg, preferably from 200 to 800 mg, preferably from 400 to 600 mg, advantageously between 450 and 550 mg, of said at least one preparation based on said at least one protein hydrolysate,
preferably said composition comprising a) and b).

9. A nutritional composition, a compound feed for animals such as a complete feed for animals or a supplementary feed for animals, a feed for animals which is aimed at specific nutritional objectives, comprising the composition according to any one of the preceding claims.

10. A veterinary medicine comprising the composition according to any one of claims 1 to 8.

11. The composition according to any one of claims 1 to 8, the nutritional composition, the compound feed for animals, the feed for animals which is aimed at specific nutritional objectives according to claim 9, the veterinary medicine according to claim 10, for its/their use(s) as a veterinary medicine, in particular in domestic animals, preferably in companion animals such as dogs, cats and/or NCAs, advantageously in companion animals such as dogs and/or cats.

12. The composition according to any one of claims 1 to 8, the nutritional composition, the compound feed for animals, the feed for animals which is aimed at specific nutritional objectives according to claim 9, the veterinary medicine according to claim 10, for its/their use(s) to prevent, regulate and/or treat, in animals, in particular in domestic animals, preferably in companion animals such as dogs, cats and/or NCAs, advantageously in companion animals such as dogs and/or cats:
fear and/or anxiety, preferably anxiety, in particular in response to chronic mild stress factors and/or
at least one behaviour disorder, preferably associated with fear and/or with anxiety, advantageously associated with anxiety.

13. The composition according to the preceding claim, wherein said at least one behaviour disorder is selected from: the act of attacking, aggressiveness, destroying, inappropriate elimination, the act of repeatedly licking a part of its body, the act of scratching itself, astasia, the act of trembling, the act of marking its territory, aberrant motor behaviour, abnormal feeding behaviour such as polyphagia or polydipsia, dysbasia, an abnormal sensory profile, an abnormal posture, an abnormal vocalisation, sleep disorders, loss of expression, loss of sociability and abnormal judgement of situations.

14. The composition according to any one of claims 1 to 8, the nutritional composition, the compound feed for animals, the feed for animals which is aimed at specific nutritional objectives according to claim 9, the veterinary medicine according to claim 10, for its/their use(s) in the improvement of learning processes in animals, in particular in domestic animals, preferably in companion animals such as dogs, cats and/or NCAs, advantageously in companion animals such as dogs and/or cats.

15. The composition according to any one of claims 11 to 14, said composition being administered to the animal in the form of at least one dose, preferably in the form of a plurality of doses, said at least one dose comprising:
a) from 0.5 to 100 IU, preferably from 20 to 80 IU, preferably from 40 to 65 IU, advantageously from 50 to 60 IU, of superoxide dismutase per kilogram of body weight of said animal, and/or
b) from 10 to 1,000 mg, preferably from 200 to 800 mg, preferably from 400 to 600 mg, advantageously between 450 and 550 mg, of said at least one preparation based on said at least one protein hydrolysate per kilogram of body weight of said animal,
preferably said dose comprising a) and b).
